(19) United States
(12) Patent Application Publication
(10) Pub. No.: US 2022/0296225 A1
(43) Pub. Date: Sep. 22, 2022

(54) SYSTEM FOR PERFORMING MINIMALLY INVASIVE SURGERY

(71) Applicant: Virtuoso Surgical, Inc., Nashville, TN (US)

(72) Inventors: Richard Hendrick, Nashville, TN (US); Evan Blum, Nashville, TN (US); Neal Dillon, Nashville, TN (US); Trevor Bruns, Clarksville, TN (US); Stephanie Amack, Nashville, TN (US); Lauren Branscombe, Nashville, TN (US)

(21) Appl. No.: 17/655,477

(22) Filed: Mar. 18, 2022

Related U.S. Application Data
(60) Provisional application No. 63/162,609, filed on Mar. 18, 2021.

(51) Int. Cl. A61B 17/00

(52) U.S. Cl. CPC A61B 17/00234; A61B 2017/00336

(57) ABSTRACT

A system for performing minimally invasive surgery includes a holding arm, a holding arm interface, an actuation unit detachably disposed on the holding arm interface, and an endoscopic sheath assembly disposed on the holding arm interface opposite the actuation unit. The actuation unit includes a component bay configured to receive a camera, lens and first and second removable and disposable cartridges. Each cartridge includes a concentric tube array extending therefrom, each array including at least one guide tube and a surgical tool disposed inside the guide tube. Numerous safety and communication features are disposed on the various components of the system to ensure failsafe operation and to prevent damage to equipment or harm to patients.

### CROSS-REFERENCES TO RELATED APPLICATIONS

This application claims priority to U.S. Provisional Patent Application No. 63/162,609, entitled “SYSTEM FOR PERFORMING MINIMALLY INVASIVE SURGERY,” filed on Mar. 18, 2021, which is pending, and which is incorporated by reference in its entirety.

### STATEMENT REGARDING FEDERALLY SPONSORED RESEARCH OR DEVELOPMENT

This invention was made with government support under R44HL140709 awarded by the National Institutes of Health (NIH). The government has certain rights in the invention.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### REFERENCE TO SEQUENCE LISTING OR COMPUTER PROGRAM LISTING APPENDIX

Not Applicable.

### BACKGROUND

The present invention relates to surgical devices and associated methods for performing surgery. More particularly, the present invention relates to tools and methods for minimally invasive surgery using concentric tube assemblies.

Minimally invasive surgery using electromechanical robots is a developing field of medicine. Conventional devices for performing minimally invasive surgery, such as endoscopes and resectoscopes, generally include a distal tip that is inserted through an incision or a natural orifice in a patient's body. The distal tip includes an optical lens which allows a surgeon to see a field of view proximate to the distal tip when placed inside the body. The endoscope will typically have a camera attached to the lens to display the field of view on an operating room monitor. In some applications the endoscope includes a camera installed on the distal tip of the endoscope. The device also includes a narrow working channel extending through the device. One or more elongated surgical tools may be inserted through the working channel. A tool such as a cutting device, a basket or a laser optic may be included on the surgical tool. The distal end of the surgical tool protrudes from the distal tip of the device, thereby allowing the surgeon to visually observe operation of the tool inside the patient's body during an operation.

Over the past few decades, it has become increasingly clear that entering the body in the most minimally invasive way possible during surgery provides tremendous patient benefit. Minimally invasive surgery is a general term used to describe any surgical procedure that enters the body without large, open incisions.

Minimally invasive surgery includes laparoscopic surgery, which uses a tube to deliver visualization (i.e. an endoscope) and view the surgical field and long, rigid instruments that pass through small ports in the body. In conventional laparoscopic surgery, the endoscope is usually used only for visualization of the surgical field and does not have tools passing through it. The tools are pivoted outside of the body and through the incision port to provide instrument manipulation at the surgical site. The tool manipulation in laparoscopic surgery is created by pivoting long, rigid shafts through ports in the body. For surgery in the insufflated abdomen, chest cavity, pelvis or any other anatomical working volume with sufficient space, this concept often provides an excellent minimally invasive solution for delivering instrument manipulation. However, when the surgical site is down a long, narrow channel, the ability to pivot these long, rigid shafts diminishes. The tool's manipulation ability drops off sharply as access channels become longer and/or narrower.

Minimally invasive surgery also includes endoscopic surgery. While laparoscopic surgery uses endoscopes to provide visualization, endoscopic surgery differs in that the surgical instruments are passed through a working channel of the endoscope tube itself. Some examples of surgical instruments that can be used during endoscopic surgery are scissors, forceps, laser fibers, and monopolar/bipolar cautery. There are both rigid and flexible endoscopes—rigid endoscopes being used in surgeries where a straight, linear path can be taken from the outside of the body to the surgical site, and flexible endoscopes being used where winding through curving anatomy is required. Rigid endoscopes are currently used in almost every area of surgery, including but not limited to neurologic, thoracic, orthopedic, urologic and gynecologic procedures. While rigid endoscopy is currently used in surgeries all over the body, it is not without drawbacks. Tools that operate through the working channel of rigid endoscopes are similar to laparoscopic tools in that they are normally straight, rigid tools. Generally, these tools are also limited to two degrees-of-freedom motion relative to the endoscope: they can insert/retract and rotate axially. Sometimes, the surgeon may have the ability to pivot/tilt the endoscope outside of the body, which makes things particularly challenging, as whenever the endoscope moves, the field of view of the endoscope moves along with it. Also, the surgeon can only get one instrument at a time to the surgical site the vast majority of the time due to the size constraints of the working channel of the endoscope—effectively eliminating the ability for two-handed bimanual tasks. This limitation to a single tool at a time, the constantly changing field of view, limited degrees of freedom, and lack of instrument dexterity at the tip of the endoscope make endoscopic surgery a particularly challenging type of minimally invasive surgery.

Because they are particularly skilled with precision, spatial reasoning, and dexterity, electromechanical surgical robots have great potential to aid in surgical instrument manipulation and is a rapidly developing field of medicine. Surgical robots have gained widespread adoption throughout the world and have been utilized in hundreds of thousands of procedures. The majority of surgical robotic systems designed thus far that aid in instrument manipulation can be generally categorized into pivoted and flexible tools. Pivoted, laparoscopic-like systems such as the widely used da Vinci Xi robot, made by Intuitive Surgical, Inc., gain instrument manipulation in the same way that laparoscopic tools do: by tilting through a port in the body. For surgical applications where tilting or pivoting of the tools is not possible outside of the body, several groups in the research community have been developing robotic systems based on flexible elements. These systems are often referred to as continuum robots, or a continuously bending, robot with an elastic structure. There also exist concentric tube manipulators, which are a class of miniature, needle-sized continuum robot composed of concentric, elastic tubes. Concentric tube robots appear promising in many kinds of minimally invasive surgical interventions that require small diameter robots with articulation inside the body. Examples include surgery in the eye, ear, sinuses, lungs, prostate, brain, and other areas. In most of these applications, higher curvature is generally desirable to enable the robot to turn “tighter corners” inside the human body and work dexterously at the surgical site. In the context of endoscopic surgery, the precurvatures of the concentric tubes determine how closely the manipulators can work to the tip of the endoscope, which is very important during endoscopic surgery.

With traditional endoscopic procedures, surgeons typically hold the endoscope in one hand and the endoscopic instrument in the other, making it generally not possible for the surgeon to simultaneously manipulate two instruments. Due to the human error aspect, whenever the surgeon needs to swap one endoscopic instrument out for another, it can result in awkward and potentially dangerous endoscope movements. Surgeons often, however, need the ability to accurately and simultaneously manipulate two instruments in certain situations—especially when trying to grasp, manipulate, and cut material precisely. Even where endoscopes can accommodate more than one tool simultaneously, the tools can only be oriented straight out and parallel to one another, which prohibits truly collaborative work between the tools. Surgeons can greatly benefit from the increased precision, dexterity, and vision that robotic surgery systems offer, but such conventional systems are limited in their manipulability.

Conventional surgical robots for performing laparoscopic and endoscopic procedures generally include a robotic arm coupled to an electromechanical actuator configured to manipulate a surgical tool disposed on its distal end. In practice, the robotic arm and the actuator must be controllable via an electronic interface. Such systems are software based and may be programmed to operate in different ranges of motion. Because the tissue workspace is relatively small compared to the overall size of such robotic systems, it is very important to ensure safeguards in the design and operation of robotic surgical systems prevent damage to equipment or injury to the patient. Many conventional surgical robots lack adequate safety systems.

Additionally, due to the overall complexity of surgical robots and the number of individual parts involved in such systems, it is vital to maintain a sterile interface between the robotic system and a surgical field. Conventional surgical robots often lack sufficient sterility features to ensure both ease of operation and a sterile environment.

Another complexity of robotic surgery involves communication between the surgeon controlling the robot and the hardware. For example, the individual components of a robotic surgery system may operate in different modes, and it is important for a surgeon to be able to quickly identify what mode a device is in, and make changes if necessary. Many conventional robotic surgery systems provide such information only on a control panel, which requires a surgeon to look away from the surgical field.

What is needed, then, are improvements in devices and methods for performing robotic surgery, and specifically for safety systems, sterility approaches, electronic interfaces and status indicators.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

A device for performing minimally invasive surgery includes a holding arm, a holding arm interface detachably mounted to the holding arm, an actuation unit detachably mounted to the holding arm interface and a sheath assembly detachably mounted to the holding arm interface opposite the actuation unit.

The system includes numerous safety and operational features to provide robust operation and to prevent damage to equipment or harm to patients.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is a perspective view of an embodiment of a system for performing minimally invasive surgery.

FIG. 2 is a perspective view of an embodiment of a system for performing minimally invasive surgery.

FIG. 3 is a partially exploded perspective view an embodiment of a system for performing minimally invasive surgery.

FIG. 4 is a front perspective view of an embodiment of a holding arm interface in accordance with the present disclosure.

FIG. 5 is a back perspective view the embodiment of a holding arm interface of FIG. 4.

FIG. 6 is a partially exploded view of an embodiment of a holding arm interface and sheath assembly in accordance with the present disclosure.

FIG. 7 is a partially exploded view of an embodiment of a holding arm interface in accordance with the present disclosure.

FIG. 8 is a partially exploded view of an embodiment of a brake for holding arm interface in accordance with the present disclosure.

FIG. 9 is a detail perspective view of an embodiment of a handle and interface mount on a holding arm apparatus in accordance with the present disclosure.

FIG. 10 is a front detail perspective cross-sectional view of an embodiment of a holding arm interface in accordance with the present disclosure.

FIG. 11 is a rear detail perspective cross-sectional view of an embodiment of a holding arm interface in accordance with the present disclosure.

FIG. 12 is a rear perspective view of an embodiment of a system in accordance with the present disclosure.

FIG. 13 is a perspective view of an embodiment of a cartridge in accordance with the present disclosure.

FIG. 14 is a rear perspective view of an embodiment of a system in accordance with the present disclosure.

FIG. 15 is a perspective view of an embodiment of a system in accordance with the present disclosure.

FIG. 16 is a perspective view of an embodiment of a physician input console in accordance with the present disclosure.

FIG. 17 is a perspective view of an embodiment of a top tray of a physician input console in accordance with the present disclosure.

FIG. 18 is a block diagram of one embodiment of a graphical user interface displayed on a physician input console in accordance with the present disclosure.

FIG. 19A is a front view of one embodiment of a graphical user interface instructing a user how to re-align an input control after misalignment in accordance with the present disclosure.

FIG. 19B is a front view of another embodiment of a graphical user interface instructing a user how to re-align an input control after misalignment in accordance with the present disclosure.

FIG. 20A is a side perspective view of one embodiment of a physician input console handle in accordance with the present disclosure.

FIG. 20B is another side perspective view of another embodiment of a physician input console handle in accordance with the present disclosure.

FIG. 21 is a top perspective view of one embodiment of an actuation unit in accordance with the present disclosure.

FIG. 22 is a bottom perspective view of one embodiment of an actuation unit in accordance with the present disclosure.

FIG. 23A is a perspective view of one embodiment of an input control handle in accordance with the present disclosure.

FIG. 23B is a perspective view of another embodiment of an input control handle in accordance with the present disclosure.

FIG. 24 is a side view depicting various embodiments of surgeon grips of an input control.

FIG. 25 is a cross-sectional side view of one embodiment of an input control handle in accordance with the present disclosure.

FIG. 26 is a perspective cross-sectional view of one embodiment of an input control handle in accordance with the present disclosure.

FIG. 27A is a perspective view of a draped physician input console in accordance with the present disclosure.

FIG. 27B is a perspective view of one embodiment of a draped input control in accordance with the present disclosure.

FIG. 28 is a perspective view of one embodiment of an articulated holding arm base in accordance with the present disclosure.

FIG. 29 is a perspective view of one embodiment of an articulated holding arm base in an operating table-mounted configuration in accordance with the present disclosure.

FIG. 30 is a schematic block diagram of one embodiment of a control system for a safety supervisor in accordance with the present disclosure.

FIG. 31 is a schematic block diagram of one embodiment of certain working elements of a cartridge sensing subsystem in accordance with the present disclosure.

FIG. 32 is a perspective view of a draped actuation unit in accordance with the present disclosure.

FIG. 33A is a perspective view of one embodiment of the tip of a concentric tube manipulator in accordance with the present disclosure.

FIG. 33B is a perspective view of another embodiment of the tip of a concentric tube manipulator in accordance with the present disclosure.

FIG. 34 is a perspective view of one embodiment of an optic support guide in accordance with the present disclosure.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

### Overall System

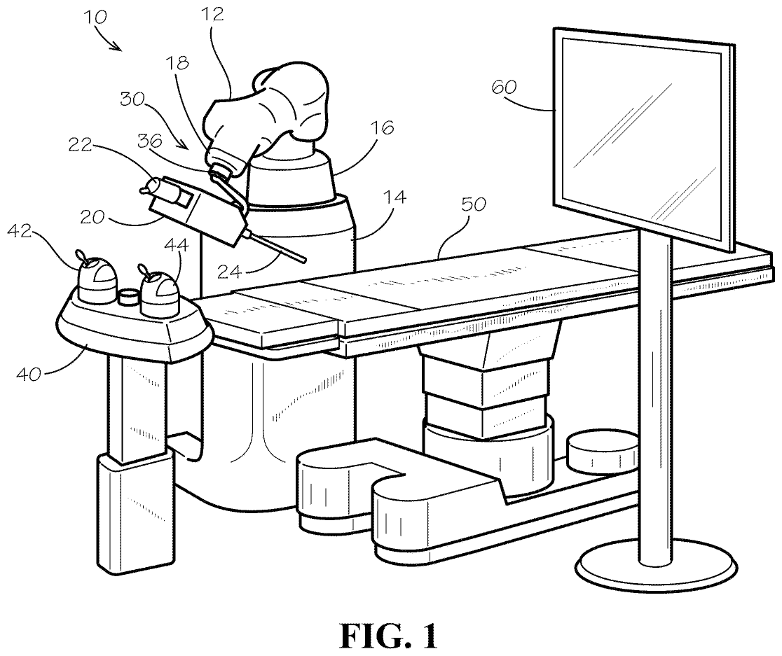

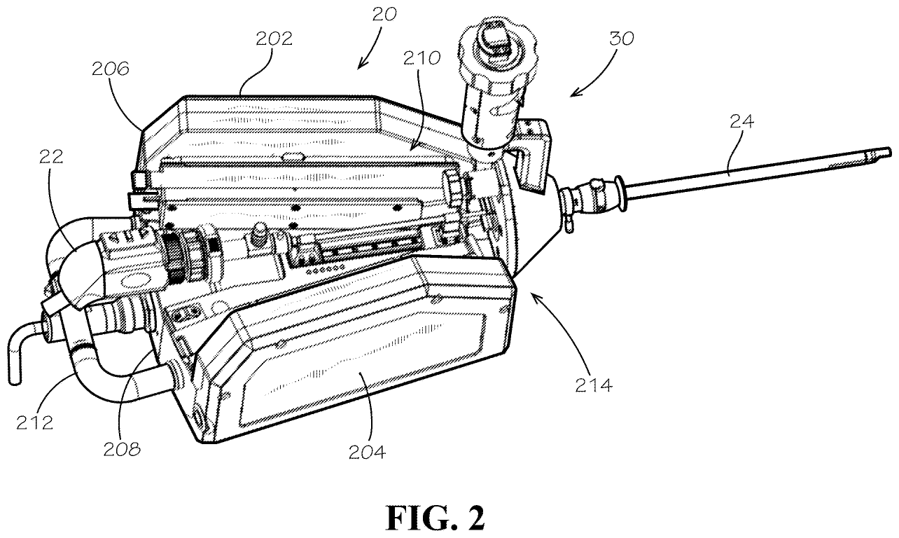

Referring to FIG. 1 and FIG. 2, the present disclosure provides a robotic system 10 for performing minimally invasive surgery. The system 10 includes a holding arm 12 mounted on a base 14. The holding arm 12 may include an articulated holding arm. The holding arm 12 may include a robotic arm or may include a passive arm. The holding arm 12 may provide assistance in manipulating the holding arm 12. For example, the system 10 may include a passive counterbalance or may include one or more motors that may provide gravity or dynamic compensation. In some embodiments, the holding arm 12 may be mounted directly to an operating table 50 rather than on the base 14. In some embodiments, the base 14 may provide a vertical/adjustable degree-of-freedom so that the holding arm 12 may be amendable to an array of patient positioning orientations. The holding arm 12 is configured to provide multiple degrees of freedom for controlling the position and angular orientation of a surgical apparatus in three-dimensional space in a surgical field, for example above the operating table 50 as shown in FIG. 1. The holding harm 12 is specifically configured for at least the applications set forth in this disclosure. In some embodiments, the holding arm 12 is mounted on an inclined wedge 16 which is secured to base 14. Wedge 16 provides enhanced positioning of the surgical apparatus over operating table 50 in some embodiments. In other embodiments, holding arm 12 is mounted directly on base 14. Base 14 may be stationary or mobile.

An actuation unit 20 is positioned on the system 10 to provide control of one or more instruments for performing a minimally invasive surgical procedure. In some embodiments actuation unit 20 includes a concentric tube assembly 24 configured for endoscopic surgery. The actuation unit 20 may accept insertable and exchangeable instrument cartridges 410 that may control the concentric tube assembly 24 and their attached tools. A camera 22 is also disposed on actuation unit 20 for real time observation on display 60 of the surgical field at the distal end of the concentric tube assembly 24 during an operation. The optic or telescope 260 may provide an optical path and light to the surgical site through the concentric tube assembly 24 and an interface for camera 22 attachment at an eyepiece. In further embodiments, the features disclosed herein may readily be implemented on robotic systems for performing minimally invasive laparoscopic surgery.

A holding arm interface (HAI) 30 connects actuation unit 20 to holding arm 12. Holding arm interface 30 includes a mechanical linkage between the actuation unit 20 and the holding arm 12. In some embodiments, an interface mount 36 is disposed on the upper end of the holding arm interface 30. Interface mount 36 mechanically engages a corresponding arm mount 18 positioned on the distal end of holding arm 12. The engagement between interface mount 36 and arm mount 18 includes both mechanical and electrical interfaces in some embodiments.

A physician input console 40 is directly or indirectly connected to the actuation unit 20. Physician input console 40 includes first and second input controls 42, 44 configured for controlling one or more surgical tools disposed on the actuation unit 20. Holding arm interface 30 may also include one or more electronic interfaces linking actuation unit 20 and physician input console 40 in some embodiments.

System 10 includes numerous features to provide precise control, safety, sterility and communications for performing surgical operations. Many of the safety features are provided to ensure the system components are not damaged during use or transport, and other safety features are provided to protect a patient and healthcare workers before, during or after a surgical procedure. The safety features described herein are independent and may be employed as individual features, or in combination with each other as part of a comprehensive surgical system.

Referring to FIG. 2, an embodiment of an actuation unit 20 mounted to a holding arm interface 30 is shown. Actuation unit 20 includes a base 202 having a first side wall 204 and a second side wall 206 spaced from the first side wall 204. A platform 208 spans between the first and second side walls 204, 206, forming a U-shaped frame. A component bay 210 is defined above the platform 208 between the first and second side walls 204, 206. A platform handle 212 extends rearwardly from the platform 208 to allow a user to manually reposition the actuation unit 20 during use. Platform handle 212 may also be used for engagement or disengagement of actuation unit 20 with holding arm interface 30.

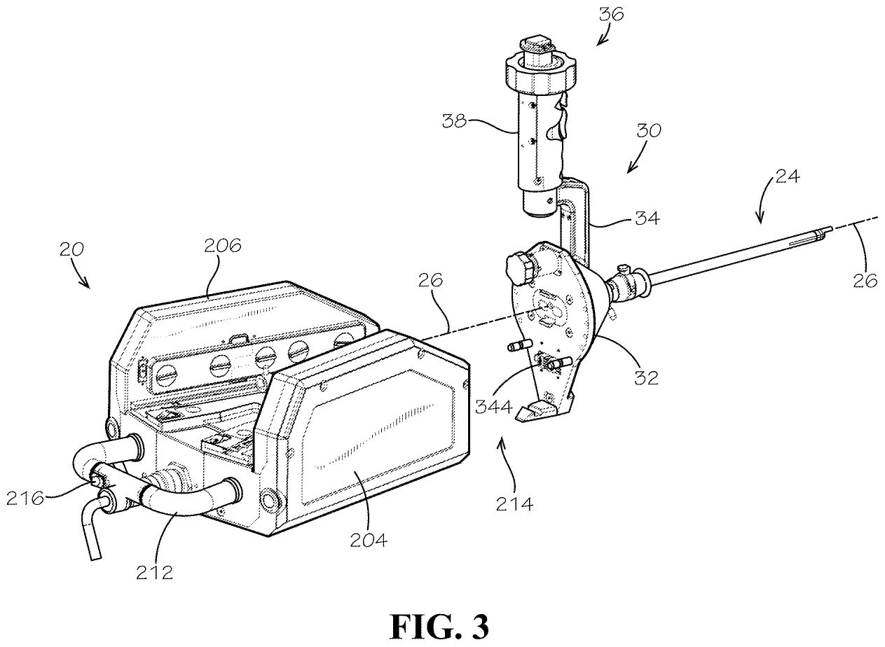

As shown in FIG. 3, holding arm interface 30 includes a body 32 and a bracket 34 extending upwardly from the body 32. An interface mount 36 is disposed on the upper end of holding arm interface 30 for attachment to arm mount 18 on holding arm 12. In some embodiments, an interface handle 38 is positioned on holding arm interface 30 between body 32 and interface mount 36. Interface handle 38 provides a location for a user to grip holding arm interface 30 to manually steer concentric tube assembly 24 relative to a tissue workspace.

### Holding Arm Interface (HAI) Slide Out Fail Safe

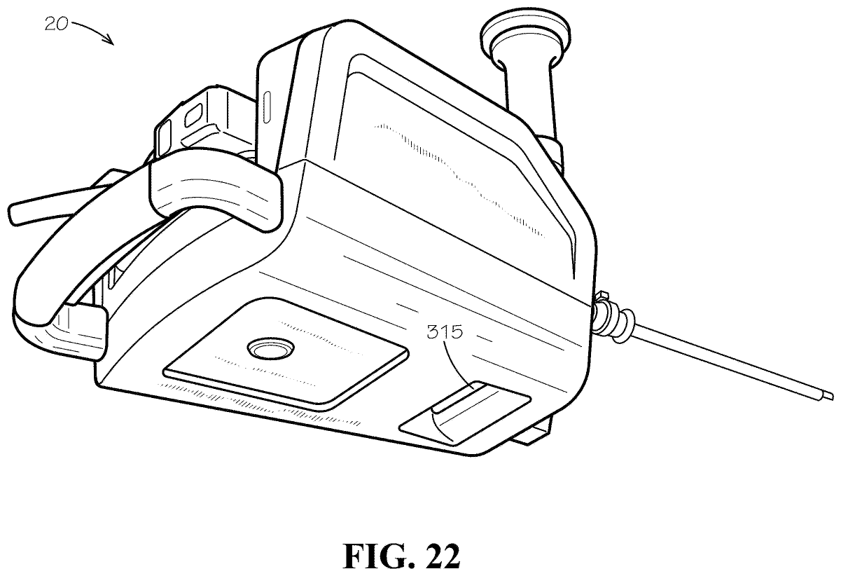

A detachable joint 214 is provided between actuation unit 20 and holding arm interface 30, as shown in FIG. 2 and FIG. 3. As such, actuation unit 20 may be selectively detached from holding arm interface 30. Such detachment may occur when holding arm interface 30 is rigidly secured to the holding arm 12. This modular configuration allows actuation unit 20 to be physically disengaged from holding arm interface 30 during transport or even during a surgical procedure. For example, during an operation, it may be necessary to quickly withdraw one or more structures from the patient's tissue workspace. By providing a releasable attachment between actuation unit 20 and the holding arm interface 30, the actuation unit 20 along with its subassemblies may be quickly withdrawn in a direction away from the patient. In one embodiment, the actuation unit 20 may include a user latch release 315 (seen in FIG. 22). The user latch release 315 may be disposed on a bottom portion of the actuation unit 20. A user may pull the user latch release 315 to release a latch and decouple the holding arm interface 30 from the actuation unit 20.

Another feature of the present disclosure provides an actuation unit 20 that is attached or detached along longitudinal insertion axis 26, which is co-linear with the travel axis of the endoscopic tools housed in tube assembly 24. Inserting or removing the actuation unit 20 and its components along the same longitudinal axis as the endoscopic axis provides enhanced safety, as side-to-side motion within the tissue workspace is minimized, and the potential for trauma to surrounding tissue is greatly reduced. Any other decoupling designs that do not restrict travel to longitudinal insertion axis 26 may be more dangerous and could lead to unacceptable risk to the patient, or damage to the equipment.

Another feature of the present disclosure provides an actuation unit 20 that may be disengaged from the system with or without power. The detachable joint 214 utilize mechanical disconnects that may be mechanically released in the event power is lost or a malfunction occurs. This additional safety feature helps prevent scenarios where one or more surgical tools may inadvertently be held in place in the patient's body during a loss of power.

In some embodiments, a release switch 216 is positioned on platform handle 212. A user may operate release switch 216 to release the mechanical engagement between actuation unit 20 and holding arm interface 30. Release switch 216 may include a mechanical or an electrical switch in various embodiments.

Holding arm interface 30 and actuation unit 20 are also configured such that electrical interface between the two may be easily disconnected during separation of detachable joint 214. For example, in some embodiments, holding arm interface 30 includes an electrical connector 344 forming one or more pin sockets positioned to receive a corresponding connector on the distal end of actuation unit 20. When actuation unit 20 is detached from holding arm interface 30, the electrical connector 344 on holding arm interface 30 disengages from the corresponding connector on actuation unit 20 along the same direction of travel as the disengagement motion.

### Holding Arm Interface (HAI) Safety Critical Signal

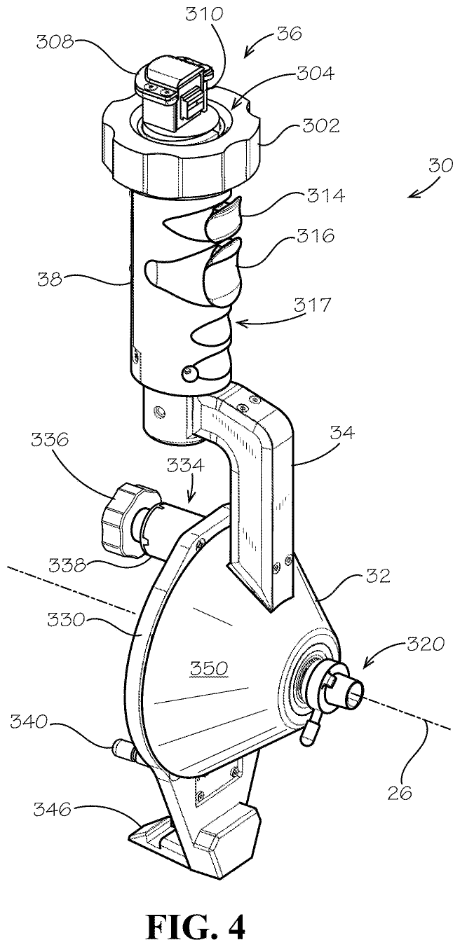

Referring to FIGS. 4-6, holding arm interface 30 provides an electro-mechanical linkage between holding arm 12 and actuation unit 20. Holding arm interface 30 includes an interface body 32 that is configured to receive actuation unit 20. A bracket 34 extends upwardly from interface body 32, and an interface mount 36 is disposed on the upper end of holding arm interface 30. Interface mount 36 attaches to a corresponding arm mount 18 on arm 12. Interface mount 36 includes a mechanical engagement with arm mount 18 to securely fix holding arm interface 30 in place at the distal end of the holding arm 12.

In some embodiments, holding arm 12 includes one or more sensors positioned on or near arm mount 18 configured to detect engagement with interface mount 36. Such sensors may include any suitable mechanical or electrical sensor known in the art for detecting contact or engagement with holding arm interface 30. In further embodiments holding arm interface 30 includes one or more sensors positioned on or near interface mount 36. Such sensors may include any suitable sensor known in the art for detecting contact or engagement with holding arm 12. In additional embodiments, a first sensor is disposed on holding arm 12, and a second sensor is disposed on holding arm interface 30. The first sensor is configured to detect engagement with holding arm interface 30, and the second sensor is configured to detect engagement with holding arm 12. As such, the system 10 includes redundant safety sensors that each may independently detect the presence of the opposing structure.

When the holding arm interface 30, holding arm 12, or both detect an engagement between the holding arm interface 30 and holding arm 12, a holding arm interface safety signal is generated. The HAI safety signal is received by one or more safety relays on the robotic holding arm 12. When the holding arm 12 detects the HAI safety signal, the safety relays on the holding arm 12 prevent autonomous movement of the holding arm 12. This may be achieved in a variety of different ways on the holding arm 12, including electrical, software and/or mechanical operation to limit movement of the holding arm 12. This safety feature utilizes the HAI safety signal to detect a condition when the holding arm interface 30 is attached to the holding arm 12. If such condition is detected, the holding arm 12 is rendered temporarily unable to move autonomously for as long as the holding arm interface 30 is attached. If holding arm interface 30 is disconnected, and the HAI safety signal indicates such detachment, then the holding arm 12 may resume autonomous movement.

Additionally, during such times as the holding arm interface 30 is detected to be attached to the holding arm 12, the first and second control buttons 314, 316 on the holding arm interface 30 alternate between impedance modes. These buttons are tied directly to the safety controller and safety relays of the holding arm 12 as well. This also provides a safety feature to the overall system.

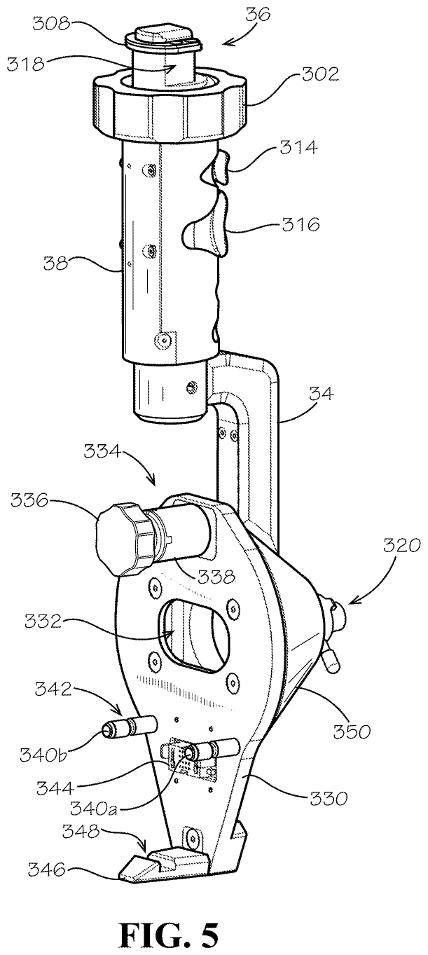

Referring to FIG. 5, the holding arm interface 30 may include a first flat surface 318 that may allow motion in a direction parallel to the first flat surface 318, which may include the longitudinal axis of an endoscope. The holding arm 12 mating coupling may include a similarly flat female receptacle. The endoscope may move along the longitudinal axis during decoupling from the holding arm 12, which may enable safe decoupling of the holding arm interface 30 from the holding arm 12, while the endoscope may still be inside of the patient. Because there are scenarios where the holding arm 12 may stop and become braked—for example during a power loss—it may be advantageous to be able to safely remove the endoscope from the patient's body in way that does not require holding arm 12 motions or that may be passive. The holding arm interface 30 may include a second flat surface (for example, on the mounting flange 308) that may allow the user to decouple the holding arm interface 30 from the holding arm 12 and allow the holding arm 12 to rest on this second flat surface under gravity, without having to hold the weight of the actuation unit 20. This feature enhances safety and provides for ease-of-use benefits.

Referring further to FIGS. 4-6, holding arm interface 30 includes a number of features that provide enhanced operability and safety. As shown in FIG. 4, holding arm interface 30 includes an interface mount 36 that provides a mechanical and an electrical connection to holding arm 12. Interface mount 36 in some embodiments includes a rotating collar 302 with a threaded configuration 304. An electrical interface 310 is positioned above the threaded mount positioned to engage a corresponding electrical contact on the holding arm 12. A mounting flange 308 extends laterally from the mount in some embodiments to provide mechanical engagement with corresponding structure on the arm mount 18 in some embodiments.

Interface handle 38 is located below the interface mount 36 and includes a grip region having finger grooves 317 in some embodiments. Interface handle 38 includes a cushioned material such a plastic, foam or rubber grip in some embodiments. Interface handle 38 includes first and second control buttons 314, 316 which may be configured for different control functions, such as a release of the arm 12 to allow manual manipulation or repositioning of the holding arm interface 30. First and second buttons 314, 316 may also control other features of the device in different embodiments. Bracket 34 connects interface handle 38 to body 32.

### Sheath Detachment

Body 32 is configured for detachable engagement with actuation unit 20 on its proximal side and detachable engagement with tube assembly 24 on its distal side facing the patient. A sheath mount 320 is positioned on the distal side of body 32 facing toward the patient and away from the actuation unit 20. Sheath mount 320 provides a detachable joint between holding arm interface 30 and the tube assembly 24 which houses the endoscopic channels which guide insertion and retraction of the endoscopic tubes and instruments, inner sheath and outer sheath. Sheath mount 320 provides a releasable mechanical engagement that may be quickly released to allow the tube assembly to be detached along and removed along the longitudinal insertion axis 26.

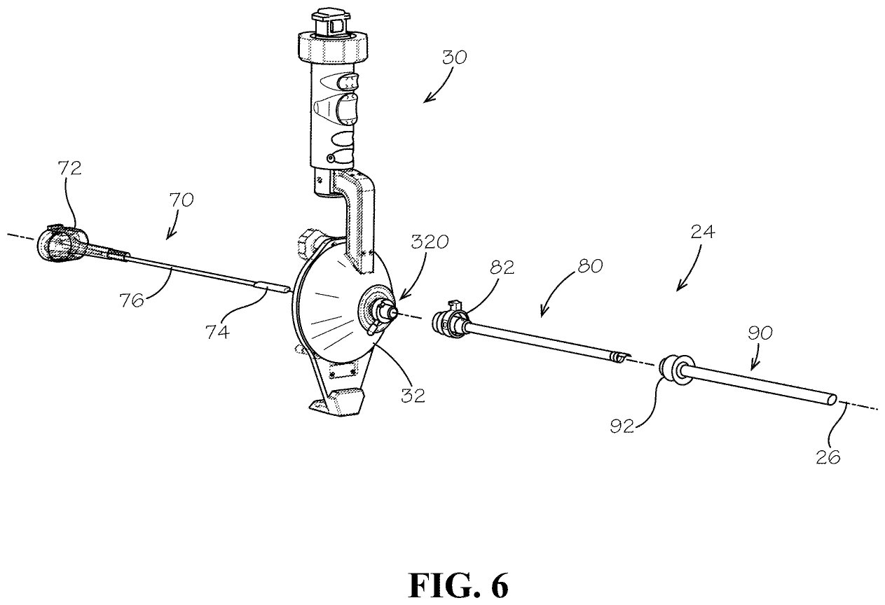

Referring to FIG. 6, inner sheath 80 includes an inner sheath latch 82 that mechanically engages with sheath mount 320 on holding arm interface 30. Inner sheath latch 82 slides onto sheath mount 320 and rotates into a locking position in some embodiments. As such, inner sheath latch 82 forms a rigid linkage and seal between sheath mount 320 and inner sheath 80. Inner sheath 80 is inserted onto sheath mount 320 along longitudinal insertion axis 26, which provides an additional measure of safety, as travel of the components is limited to a common axis.

Outer sheath 90 slides over inner sheath 80, and an outer sheath latch 92 engages inner sheath latch 82 to secure outer sheath 90 to inner sheath 80, thereby forming a rigid linkage and a seal between inner sheath 80 and outer sheath 90 in some embodiments. In one embodiment, the endoscopic sheath assembly includes the inner sheath 80 and the outer sheath 90.

Channel assembly 70 includes first and second tubular channels 76 that each receive a concentric tube assembly 24 that houses endoscopic instruments. Channel assembly 70 includes a proximal end 72 and a distal end 74. Channel assembly 70 may be inserted into inner sheath 80 through a passage in holding arm interface 30 along longitudinal insertion axis 26. This provides an additional measure of safety, as travel of the components is limited to a common axis.

By providing a detachable interface between the sheaths and the holding arm interface 30, a safer configuration is achieved. If the sheaths were a permanent fixture to the actuation unit 20 or holding arm interface 30, insertion of the tools into the patient would be more dangerous and challenging due to the additional mass of the robot and the actuation unit 20. The present disclosure provides embodiments that permit manual insertion of the outer sheath, and decoupling of the inner sheath from the remainder of the actuation unit 20. The decoupling of the sheaths may also enable use of existing conventional instruments during atraumatic insertion, eliminating the need for special tools for inserting the robotic system 10 into the patient.

### Rotational Degree of Freedom

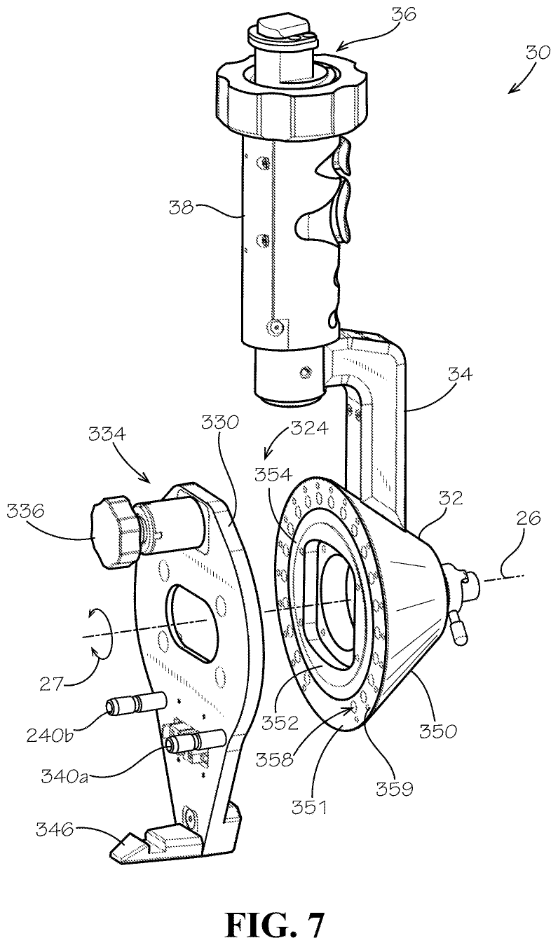

Another feature of the present disclosure provides a system for performing robotic surgery with a rotational degree of freedom about the longitudinal insertion axis 26. Referring to FIG. 7, holding arm interface 30 includes a rotating joint 324 that allows rotation of actuation unit 20 relative to body 32, bracket 34, interface handle 38 and interface mount 36. In other words, the actuation unit 20 and tube assembly 24 may be rotated about longitudinal insertion axis 26 while the remainder of the holding arm interface 30 remains rigidly fixed to holding arm 12. This rotational degree of freedom allows the endoscope to spin about the longitudinal axis and enables the surgeon to look around the anatomy with a non-zero direction of view on the rod lens.

The rotating joint 324 includes a base plate 330 on the proximal side of the holding arm interface 30. Base plate 330 may be rotated relative to outer shell 350 on body 32. Outer shell 350 includes a cone-shape with a flat rear surface. A rigid funnel housing 352 is positioned inside body 32, and base plate 330 is attached to funnel housing 352 using one or more fasteners. A first bearing 352 is disposed between funnel housing 352 and outer shell 350 such that funnel housing 352 may rotate about longitudinal insertion axis 26 inside outer shell 350 while outer shell 350 remains stationary. As such, when base plate 330 is secured to funnel housing 352, base plate 330 may also rotate bi-directionally 27 about longitudinal insertion axis 26 simultaneously with the rotation of funnel housing 352. When actuation unit 20 and its corresponding components are secured to base plate 330 via mounting posts 340a, 340b and bottom latch 346, actuation unit 20 also rotates together with base plate 330 and funnel housing 352, thereby allowing rotation of the camera lens and endoscopic concentric tube arrays extending through the tube assembly into the tissue workspace.

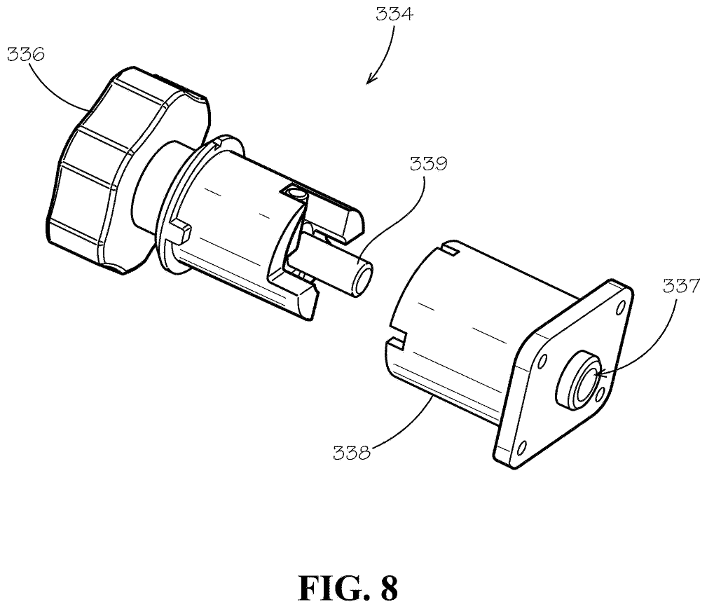

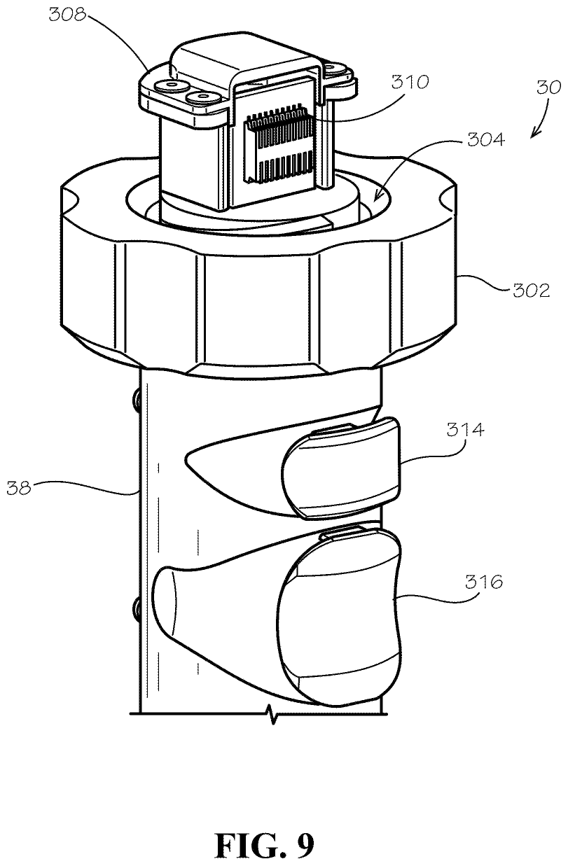

When an operator rotates the actuation unit 20 to a desired angular orientation via rotating joint 324 on holding arm interface 30, it may be desirable to maintain the new angular orientation for a period of time. To achieve this, the present disclosure provides a brake 334 which allows the base plate 330 to be locked at a desired angular orientation relative to body 32. Brake 334 includes a brake knob 336 attached to a brake pin 339, shown in FIG. 8. A brake housing 338 is secured to the base plate 330, and a brake pin orifice 337 allows brake pin 339 to be selectively extended to engage a corresponding brake pin socket 358 defined on the surface 351 of body 32 facing base plate 330. When the brake 334 is engaged, brake pin 339 extends into a brake pin socket 358 thereby locking base plate 330 in a desired angular orientation. Brake 334 may be released by operation of brake knob 336 in a push and twist motion thereby withdrawing brake pin 339 from brake pin socket 358.

It is desirable in some applications to limit the free rotation of actuation unit 20 such that the device may not freely spin about longitudinal insertion axis 26 when brake 334 is disengaged. An angular detent assembly is provided to provide some resistance to free angular rotation of base plate 330. Angular detent assembly includes a plurality of angular detent recesses 359 defined on the rear-facing surface 351 of body 32. Each angular detent recess 359 is angular aligned with a brake pin socket 358 in some embodiments such that brake pin 339 will be biased in alignment with a brake pin socket 358 at each angular position.

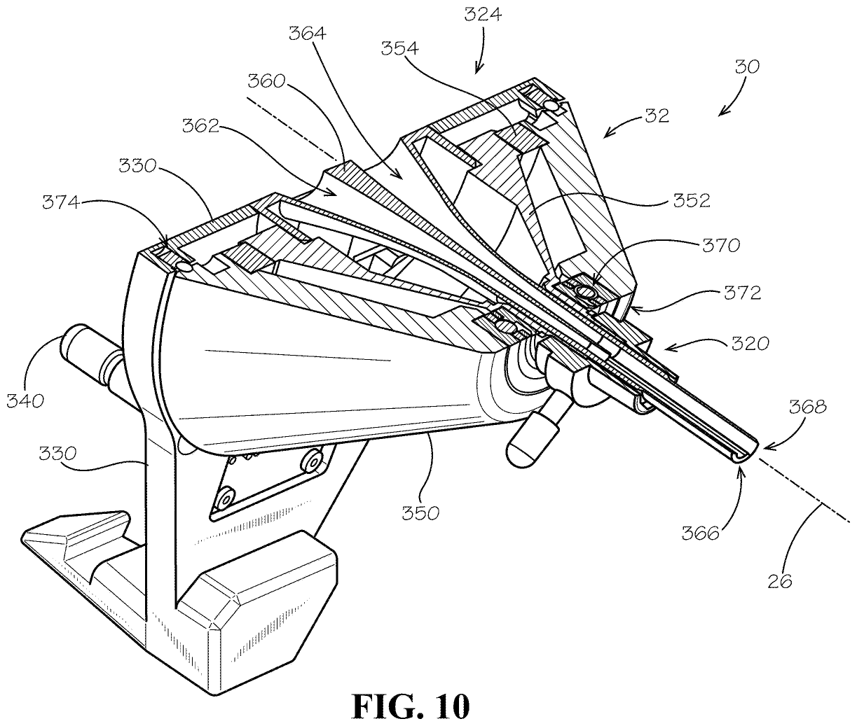

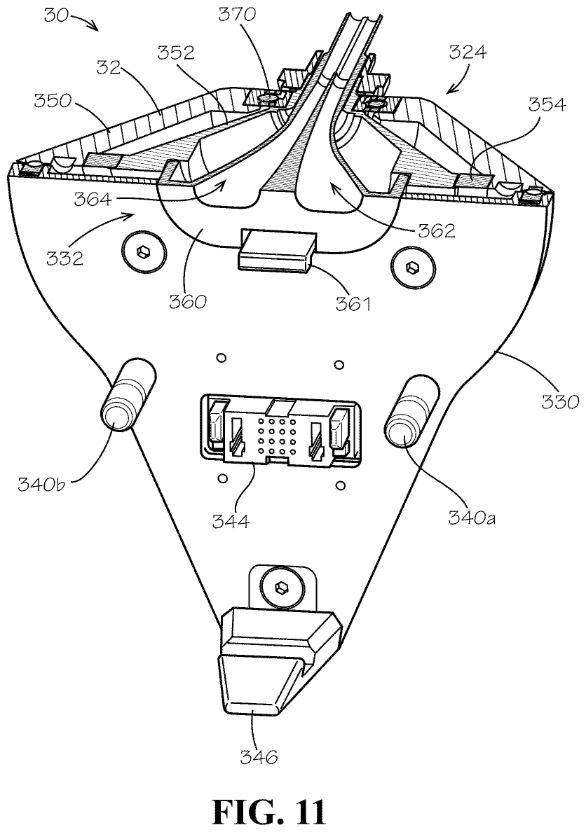

Referring to FIGS. 10 and 11, angular detent assembly 374 is positioned circumferentially around the outer perimeter of the body 32 defining a number of pre-determined angular stops. As base plate 330 is rotated relative to shell 350, a detent ball or detent post is biased toward shell 350 and slides into its corresponding recess 359. The force applied by the detent structure is configured such that it does not lock base plate 330 relative to shell 350, but rather provides a temporary engagement that operates to facility easy alignment of the brake pin 339 with a brake pin socket while also limiting the unrestricted rotation of the assembly.

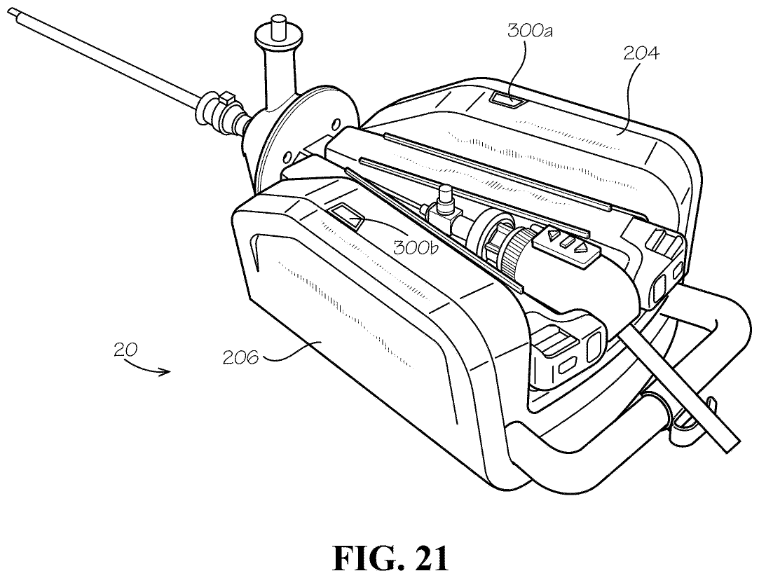

In some embodiments, an angular locking plunger may be provided by a solenoid or another actuation mechanism. In embodiments where the angular locking plunger is actuated, the user's input to lock or unlock this angular degree-of-freedom may be placed remotely on the actuation unit 20. In one embodiment, referring to FIG. 21, one or more buttons 300a, 300b for unlocking the angular rotation degree-of-freedom may be located on a side wall 204, 206 of the actuation unit 20. In some embodiments, the control algorithm may require multiple buttons 300a, 300b being depressed simultaneously to unlock this degree-freedom, such as buttons 300a and 300b being pressed simultaneously. This provides an added measure of safety so that this degree-of-freedom is not accidentally unlocked during use.

Also shown in FIGS. 10 and 11, rotating joint 324 is configured such that a funnel housing 352 is supported by a first bearing 354 on the proximal side of the body 32, and also by a second bearing 370 on the proximal side of the body 32. As such, funnel housing 352 may rotate axi-symmetrically about longitudinal insertion axis 26 without any wobble or lateral motion.

A funnel 360 is inserted into funnel housing 352 along longitudinal insertion axis 26 via access opening 332 on base plate 330, shown in FIG. 5. Funnel 360 may include first and second tapered channels that allow concentric tube assemblies housing surgical tools to be inserted longitudinally into the channel assembly 70 and down the length of tube assembly 24 toward a patient. First and second channels 362, 364 each include a narrowing taper as the channel advances toward the patient, thereby centering each concentric tube assembly 24 into is corresponding channel. The distal end of funnel 360 includes first and second channel sockets 366, 368 each dimensioned to receive a corresponding tubular channel of channel assembly 70. Inner sheath 80 is configured to slide onto the distal end of funnel 360 and engage sheath mount 320. Sheath mount 320 is rigidly secured to the forward end of funnel housing 352 such that sheath mount 320 rotates with rotation of funnel housing 352 at the forward rotating joint 372 when base plate 330 is rotated relative to shell 350. A funnel latch 361 is disposed on the rear end of funnel 360 to secure funnel 360 in axial position relative to holding arm interface 30.

As actuation unit 20 is rotated relative to holding arm interface 30 about rotating joint 324, it is desirable to index the degree of angular rotation so that a surgeon understands the direction and degree of angular rotation at all times. To achieve this, the present disclosure provides an angular sensor on the holding arm interface 30 that detects the angular position of the base plate 330 relative to shell 350 in some embodiments. The angular sensor provides a rotation signal, and a graphic indicator representative of the rotation signal is presented on the display 60. The indicator includes a compass in some embodiments showing the direction and degree of rotation of the actuation unit 20 relative to the holding arm interface 30.

### Flat Head Cartridge Interface

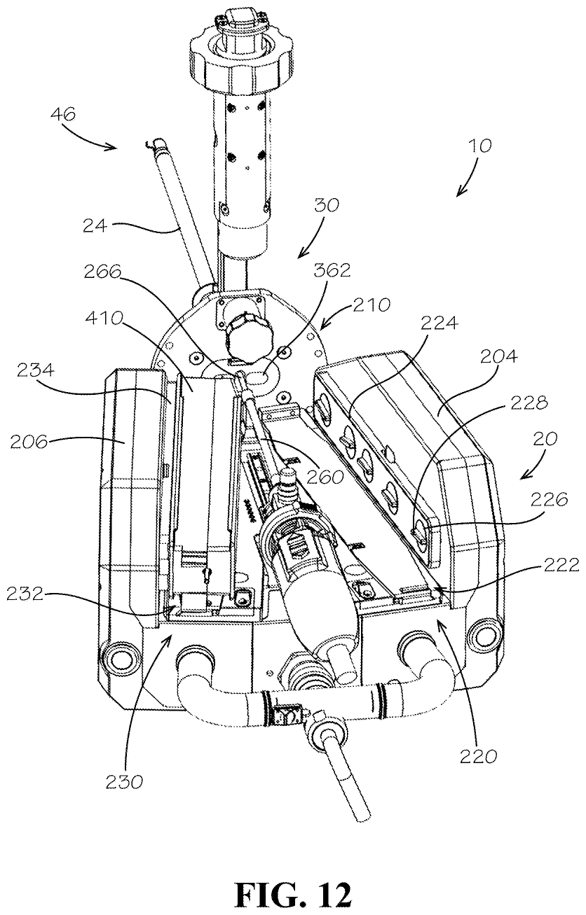

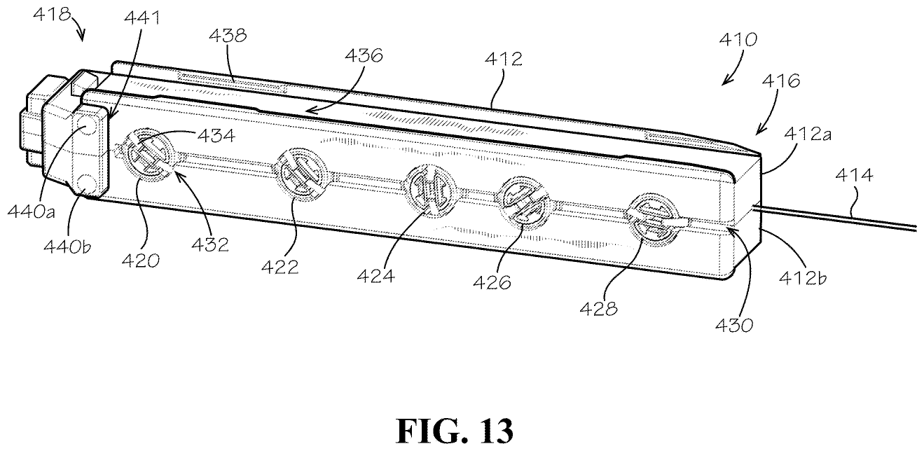

Referring to FIGS. 12-13, the present disclosure provides a removable instrument cartridge 410 that includes a concentric tube array 414 extending form the distal end of the cartridge 410. Each concentric tube array 414 includes one or more tubes for orienting a surgical tool, and one or more surgical tools 46 extending through the tube and out of the distal end of the tube assembly 24 into a patient's body during surgery. Each cartridge 410 is configured with a unique surgical tool 46 housed within a concentric tube array 414. The surgical tool 46 and one or more concentric tubes in the concentric tube array 414 may be individual manipulated via a set of gear linkages inside each cartridge 410. For example, a guide tube in the concentric tube array 414 may be axially translated relative to cartridge 410 and also rotated about its longitudinal axis relative to cartridge 410. Similarly, a surgical tool 46 housed inside the guide tube may be independent translated axially and also rotated.

Each cartridge 410 includes a plurality of independent cartridge coupling interfaces, including first, second, third, fourth and fifth cartridge coupling interfaces 420, 422, 424, 426, 428. Each cartridge coupling interface may be rotated to control an individual degree of freedom in concentric tube array 414. For example, first cartridge coupling interface 420 may be used to control axial translation of a guide tube. Second cartridge coupling interface 422 may be used to control rotation of the guide tube. Third cartridge coupling interface 424 may be used to control axial translation of the surgical tool 46. Fourth cartridge coupling interface 426 may be used to control rotation of the surgical tool 46. These are just examples, and each cartridge 410 may be configured for a customized application depending on the type of surgical tool 46 employed in concentric tube array 414.

Each cartridge coupling interface includes a coupling slot 432, and cartridge 410 includes a cartridge slot 430. When each coupling slot 432 is aligned with cartridge slot 430, a continuous linear slot is formed along the length of cartridge 410. However, if any individual coupling slot 432 is misaligned relative to cartridge slot 430, the continuous linear slot along the length of the cartridge 410 is obstructed.

During use, each cartridge coupling interface is controlled by rotation. Referring to FIG. 12, actuation unit 20 includes a first cartridge slot 220 and a second cartridge slot 230 in component bay 210. First cartridge slot 220 includes a first cartridge track 222 including a dovetailed track configured to engage a corresponding dovetail track 436 including one or more cartridge flanges 438 in each cartridge outer surface, shown in FIG. 13. During use, a cartridge 410 may be aligned such that its concentric tube array 414 is inserted into first funnel channel 362 and advanced forward, causing concentric tube array 414 to be fed into the funnel 360, and on into the tube assembly 24 down the longitudinal axis toward the tissue workspace. As the cartridge 410 and tube array 414 advance forward, the dovetail track 436 on the cartridge 410 slides into the first track 222 in first cartridge slot 220.

From this position, the cartridge 410 may only continue forward into its desired position if the cartridge coupling slots 432 are aligned with cartridge slot 430, forming an unobstructed slot down the length of the cartridge. This is because the actuation unit 20 includes a plurality of actuation couplings 226 that each correspond to a cartridge coupling 420, 422, 424, 426, 428. For example, a first actuation coupling 226 includes a linear flange 228 protruding into the first cartridge slot 220. The flat head linear flange 228 is dimensioned to slide in the cartridge slot 430 and to also slide through each cartridge coupling slot 432 as the cartridge 410 advances along its track. However, if the linear flange 228 comes to a cartridge coupling that is misaligned, the cartridge 410 is not permitted to advance further along the track. This safety feature prevents insertion of a cartridge that is not properly configured for an initial condition with respect to the concentric tube array 414. For example, each concentric tube array 414 has a desired initial condition for the distal end. This is to ensure the concentric tube array 414 can be inserted through the tube assembly 24 without snagging or becoming damaged, and also to ensure patient safety by ensuring any surgical tool 46 is in a retracted position in its initial condition. However, if a cartridge coupling were to be inadvertently rotated, such rotation might cause misalignment of the concentric tube array 414 from its desired initial condition. The present disclosure provides a flat head flange alignment between the cartridge couplings and actuation couplings to prohibit insertion if either coupling side has any single member that is misaligned away from the initial condition.

Referring further to FIG. 12, once a cartridge 410 is inserted fully in its cartridge slot, the cartridge forward end 416 reaches a travel stop that limits further forward travel, and the cartridge locks into place using a cartridge latch 418. Cartridge latch 418 engages a corresponding latch on the first track 222, thereby mechanically securing the cartridge in place. During surgery, a cartridge 410 may be retracted from the actuation unit 20 by releasing the cartridge latch 418 and pulling the cartridge rearwardly away from the actuation unit 20.

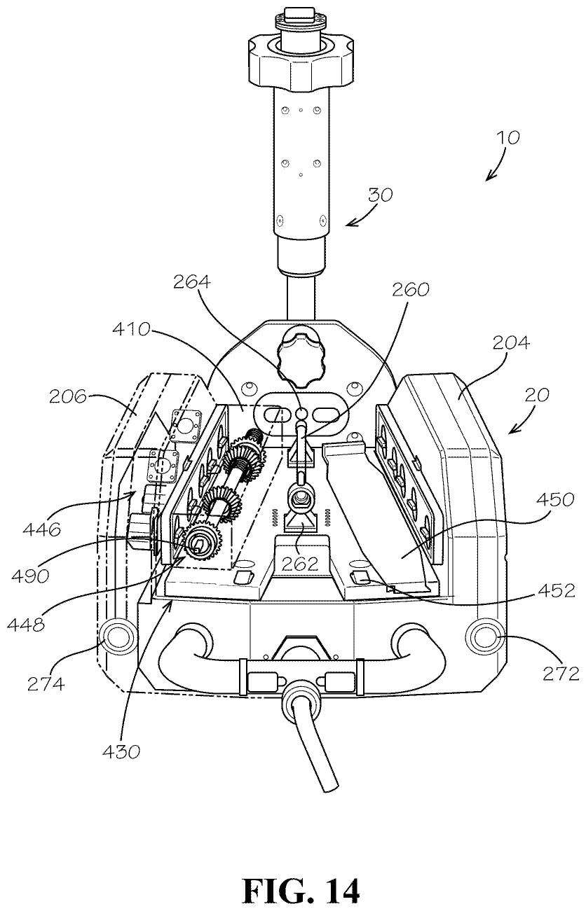

Referring to FIG. 14, each cartridge slot 220, 230 includes a corresponding motor pack housed with the adjacent side wall 204, 206. When a cartridge 410 is properly installed on actuation unit 20, each actuation coupling engages a corresponding cartridge coupling such that each coupling flange 228 is received in a corresponding cartridge coupling slot 432 on the cartridge 410. From this position, independent drive motors in each of first and second motor packs on first and second side walls 204, 206 may be operated to begin rotation of the engaged couplings. As an example, in FIG. 14 a cartridge 410 is installed in the second cartridge slot 430. A motor pack 446 housed within second side wall 206 includes separate drive motors, each drive motor corresponding to an individual coupling. Each drive motor may be operated independently to control a specific coupling. Each coupling in turn drives a component in concentric tube array 414 via a gear assembly 448. By precisely controlling the rotation of the actuation couplings 226, precision control of the cartridge couplings is achieved, which translates via gears to desired and scaled down motion of the individual components within concentric tube array 414.

### Electrosurgery Interface

In one embodiment, the instrument cartridges 410 can deliver electrosurgical probes through the concentric tube assemblies 414 to cut and coagulate tissue at the surgical site. These probes may be monopolar or bipolar and may operate in fluid medium or an air medium. The bipolar probes may operate as bipolar in saline where the two sides of the circuit are provided on the same instrument, or the two instruments may each provide one side of the bipolar circuit, so that the cutting path is between the instruments. The electrosurgery instruments can be activated using a foot pedal attached directly to the electrosurgery generator. This generator may be external to the robotic system 10, or it may be included in the system 10. The foot pedal may be attached to the base 14 or the physician input console 40. The foot pedal may generate a control signal that may travel over a cable to the electrosurgery generator. The system 10 may be configured so that electrosurgery can be activated either through a first or second input control 42, 44 or via foot pedals attached to the system 10, or via foot pedals attached directly to the electrosurgery generator.

### Fail Safe Use of Flat Head Interface

One problem associated with use of the cartridge slot flat head interface is that a cartridge may not be removed if any of the cartridge couplings are misaligned with the cartridge slot 430. During use, when the couplings have been rotated, a loss of power to the actuation unit 20 could create a scenario where the couplings are not aligned with the cartridge slot 430, and the cartridge needs to be removed. If this were to occur during a surgical procedure, it could be hazardous to the patient.

The present disclosure provides a failsafe mechanism to allow removal of each cartridge, even if the couplings are not aligned. For example, each cartridge track includes a detachable dovetail base 450. When a cartridge 410 is installed on its corresponding cartridge track 222, 232, if the cartridge 410 must be removed immediately without aligning the couplings, a track release switch 452 may be operated to immediately release the detachable base 450 from the actuation unit 20. Because each cartridge is engaged with the base 450 in a dovetail configuration, the cartridge 410 and base 450 are both released together as one attached unit. This safety feature provides a failsafe in the event power is lost to the actuation unit 20 and the cartridges must be removed.

### Cartridge Identification

In some embodiments, each cartridge 410 includes one or more devices to verify proper positioning and identification of the cartridge. For example, as shown in FIG. 13, a cartridge 410 includes an integrated cartridge chipset 441 programmed with information specific to the cartridge. Each cartridge chipset 441 includes information identifying the specific cartridge such as but not limited to the cartridge ID number, cartridge manufacturing information, cartridge sterility information, and information about the concentric tube array 414 such as the surgical tool 46 and guide tube configuration. Each cartridge chipset 441 may also include information about prior use of the cartridge. Each cartridge chipset 441 may include read only memory in some embodiments, and in other embodiments, each cartridge chipset 441 includes read and write capabilities.

In some embodiments, each cartridge chipset 441 includes a radio frequency identification (RFID), (electrically erasable programmable read-only memory) EEPROM, or near-field communication (NFC) tag device configured to store information about the cartridge. Information stored on each cartridge chipset 441 may be communicated to actuation unit 20 via one or more communication interfaces 440. For example, in some embodiments, cartridge 410 includes first and second cartridge communication interfaces 440a, 440b. Each communication interface allows communication with a corresponding circuit on the actuation unit 20. Information obtained from each cartridge chipset 441 is processed by the actuation unit 20 or by a remote processor. Such information can be used to determine if a cartridge is installed properly or if the proper cartridge is installed. If the information obtained through the cartridge communication interface reveals an error, a system fault may be generated and the system will not be operational until the fault is corrected.

In some applications, each cartridge 410 is programmed via chipset 441 such that the cartridge may only be used one time, and disposed. If a cartridge that has previously been used is installed on actuation unit 20, a system fault will be generated and the cartridge may not be used.

### Optic Support Guide

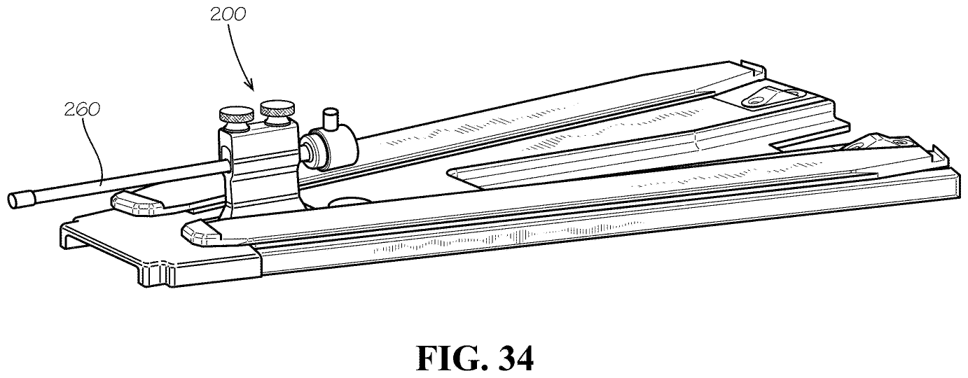

Referring back to FIG. 12 and FIG. 14, an optic support guide 260 is provided to ensure proper alignment of a rod lens into the holding arm interface 30 and the tube assembly 24. Optic support guide 260 defines the location for insertion of a rod lens 266 that travels down the length of tube assembly 24 and provides observation of the tissue workspace and surgical tool 46 in real time. Optic guide support 260 is located between first and second cartridge slots 220, 230. Optic guide support 260 includes a hollow tube mounted on a rigid standoff secured to the platform 208 on actuation unit 20. First and second cartridge tracks 222, 232 are angled, forming a clearance space between the tracks. This clearance provided by the angled orientation of the first and second cartridge tracks provides a space for positioning a camera and a lens. Without the angled orientation of first and second cartridge tracks 222, 232, there would not be sufficient room for positioning a camera and lens. However, if the first and second cartridge tracks 222, 232 were spaced in a parallel configuration, it would be nearly impossible to orient and insert each concentric tube array into its corresponding funnel channel. In some embodiments, first and second cartridge tracks 222, 232 are each angled between about five and about thirty degrees relative to the center longitudinal axis. As shown in FIG. 14, lens opening 264 is defined in the funnel, and a rod lens may be inserted through optic support guide 260 and into the lens opening 264. The bore of optic support guide 260 is axially aligned with lens opening 264 and a corresponding linear lens channel defined through the funnel. Referring to FIG. 34, in some embodiments, a manual adjustment feature 200 is provided on the optic support guide 260 which allows for the optic to be manually adjusted and locked into place. In some embodiments, this adjustment feature 200 may include a thumbscrew that tightens onto the optic support guide 260.

### Vision Controls and Adjustments

In some embodiments, the optical system may utilize a “chip-tip” imaging sensor, such as CMOS or CCD technology with integrated lighting, which may eliminate the camera 22 or the telescope 260. In one or more embodiments, the imaging sensor may be attached to the tip of a concentric tube assembly 24 such that the surgeon's view could be dynamically altered during the procedure. This may be done by a third concentric tube manipulator. In some embodiments, the robotic system 10 may provide actuation of the optical system, either the telescope 260 or the image sensor, such that the surgeon's view may be dynamically altered during the procedure. The altering of the surgeon's view may be under the direct control of the surgeon via inputs at the physician input console 40, or a control algorithm may move the image sensor in response to the surgeon's instrument movements that they convey at the first or second input controls 42, 44. This may include “eye-in-hand” techniques that enable tracking of the instruments, or a point or area between the instruments.

### Status Lights

In some embodiments, the actuation unit 20 and the holding arm interface 30 each include status lights that provide information to a user based on the light pattern, light color, light duration. For example, as shown in FIG. 14, a first status light 272 may indicate when first cartridge slot 220 is ready to receive a first cartridge, and a second status light 274 may indicate when second cartridge slot 230 is ready to receive a second cartridge. Such lights may also indicate when a fault has occurred with respect to a cartridge, motor or coupling.

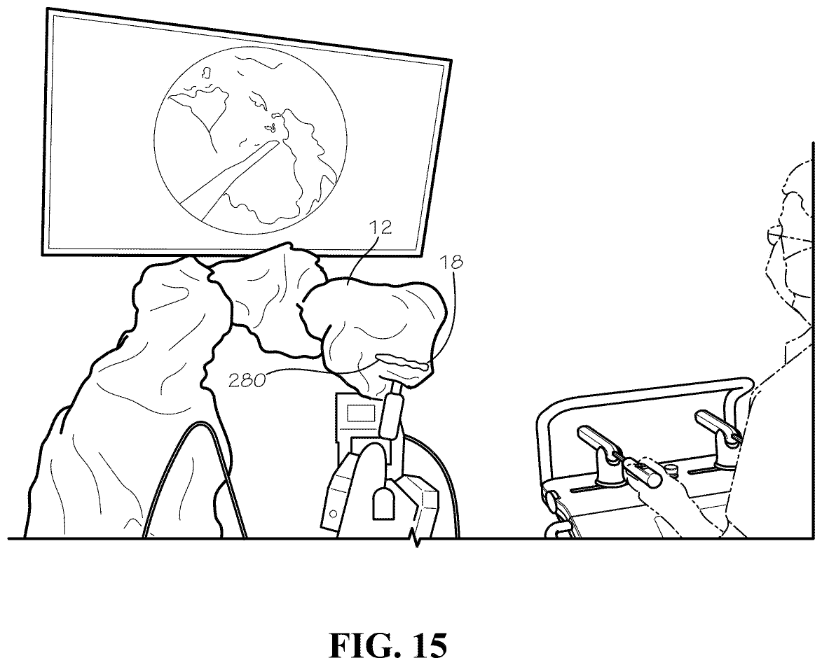

Referring to FIG. 15, an arm light 280 is disposed on arm mount 18 on holding arm 12. Arm light 280 includes a ring of lights oriented around the circumference of arm mount 18 in some embodiments. Arm light 280 may light in different colors to indicate different operational or fault states of the system. Due to the location of arm light 280, an operator may visually observe the arm light 280 to gain an immediate understanding of the state of the system. In some embodiments, arm light 280 is configured to indicate the impedance mode of the holding arm 12. Such modes can include a first color to indicate endoscope axis mode, a second color to indicate firm hold mode and a third color to indicate free motion mode. Such visual feedback mechanisms provide additional human factors safety features.

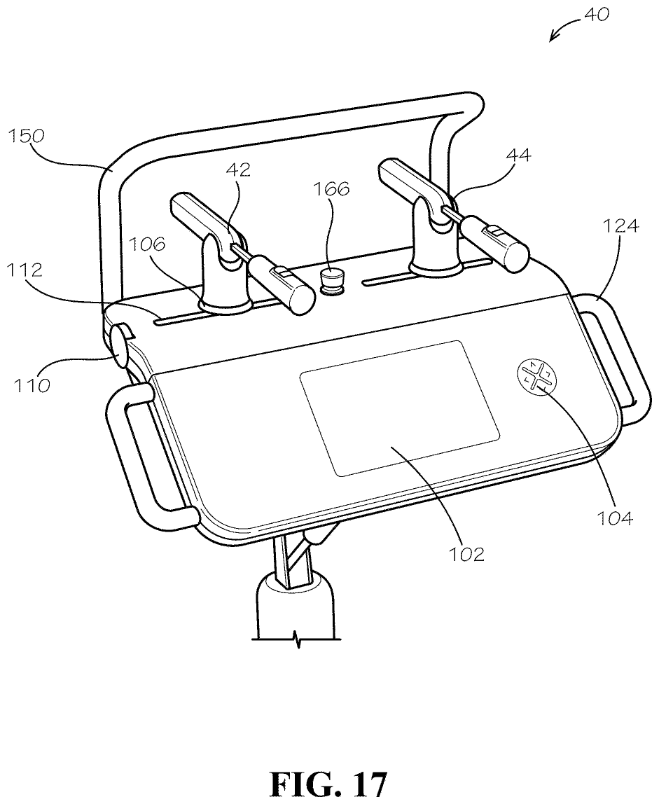

In some embodiments, the status lights 272, 274 may also be used to indicate when the actuation unit 20 can be safely removed from the patient's body. It is possible that the surgeon or operating room staff may forget to fully retract the manipulators before removing the entire actuation unit 20 and endoscope from the patient. If the manipulators were not retracted, this could cause injury to the patient during this step. One or more status lights 272, 274 on the actuation unit 20 may indicate when the actuation unit 20 can be safely removed. This information may be included as part of training the operating room staff and surgeon. Further, the status lights 272, 274 on the actuation unit 20 or the light indicators 106 of physician input console 40 (as depicted in FIG. 17) may change color to match the color of the inserted instrument cartridge 410, once the inserted instrument cartridge 410 is recognized by the robotic system 10. The color of the instrument cartridge 410 may be tool-specific, and this status light 272, 274 or indicator light 106 change may provide feedback to the user that the system 10 has recognized the correct instrument. Finally, the status lights 272, 274 or indicator light 106 may change to yellow or blue during activation of an electrosurgery instrument cartridge 410. Yellow may be the recognized color for electrosurgical cut and blue may be the recognized color for electrosurgical coagulation. In one or more embodiments, other colors may be used. This may provide feedback to the user that the system 10 is behaving as expected and allows easier user-detection of any system incorrect behavior. In some embodiments, these status light features provide a safer system 10 and allow more user awareness of the system's state.

### Embedded Motor Control

Referring back to FIG. 14, another feature of the present disclosure provides a system including an actuation unit 20 having integrated motor control and safety controller hardware on board the actuation unit 20. Some conventional robotic systems for performing surgery include remote motor control and safety controller hardware that is connected to the actuator via communication cables. However, due to the multiple degree of freedom controls presented for each cartridge in the present system, such conventional configurations are unfeasible. The present disclosure provides a system that includes motor control and safety controller hardware housed on board the actuator.

### Gripping Tool Release

Some cartridges may employ surgical tools 46 that can be actuated for gripping or grasping of tissue. Such instruments include cutting devices, gripper devices, forceps, or baskets. In the event a gripping tool 46 were engaged with tissue and a power loss occurred, it would be necessary to manually release the gripping tool 46 from the tissue such that the tool 46 could be retracted without causing trauma. The present disclosure provides gripping mechanism cartridges that include a mechanical grip release such that the grip can be released in the event of a power loss. The grip release in some embodiments, includes a manually retractable pin that will release the grip. Numerous other suitable mechanical grip release mechanisms for gripping tool 46 cartridges may be employed.

### Holding Arm Interface

As set forth above, the holding arm interface 30 includes a mechanical and electrical linkage between the holding arm 12 and the actuation unit 20. The holding arm interface 30 comprises numerous features that may be used individual or in combination with other features in a surgical system. The holding arm interface 30 is also configure to provide sterility in the surgical field by allowing a modular attachment of various components, including the endoscope sheath assembly and the actuation unit 20.

### Joint Limits and Tool Tip Safety Limits

The present disclosure provides numerous safety features to reduce risk of injury to a patient or damage to equipment. In some embodiments, the present disclosure provides a system that utilizes software-based limits to the ranges of motions of the surgical tool 46 and concentric tube array 414. Such software-based limits prevent the drive couplings from over-extending any tube array 414 or tool 46 in the tissue workspace beyond a predetermined field, even though the range of motion that actually may be mechanically achieved by the apparatus extends beyond the programmed field. By programming the control software to impose limits on the ranges of motion of the tube arrays 414 and tool 46 in the workspace, a factor of safety may be gained to prevent inadvertent damage to surrounding tissue during an operation.

In addition to the software-based limits, the cartridges themselves include hardware-based constraints on the ranges of travel available for the tube arrays 414 and tool 46. For example, the gear drive 448 includes mechanical stops on drive gears to limit the range of motion that may be imposed upon each tube array 414 and tool 46.

Another variable that defines the operational workspace for the tube arrays 414 and tool 46 includes the field of view of the camera 22 and rod lens endoscope. The rod lens provides a field of view at the distal end of the tube assembly 24. In some embodiments, the system is configured by software and/or hardware based limits to constrain motion of the tube arrays 414 and tool 46 to the space visible in the field of view of the lens. If a tube array 414 or tool 46 seeks to extend beyond the field of view, an error fault is generated and the range of motion is immediately restricted to prevent passage of the tube array 414 or tool 46 outside the field of view.

### Surgeon Workstation User Interface

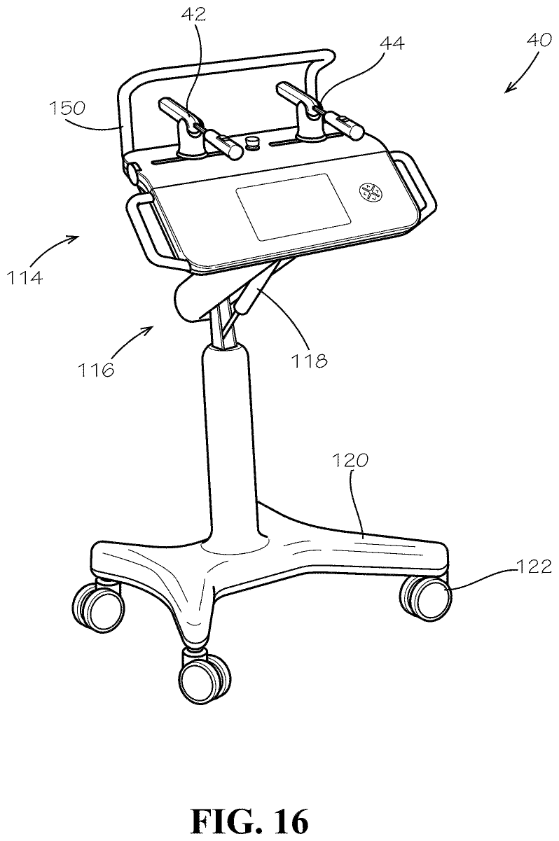

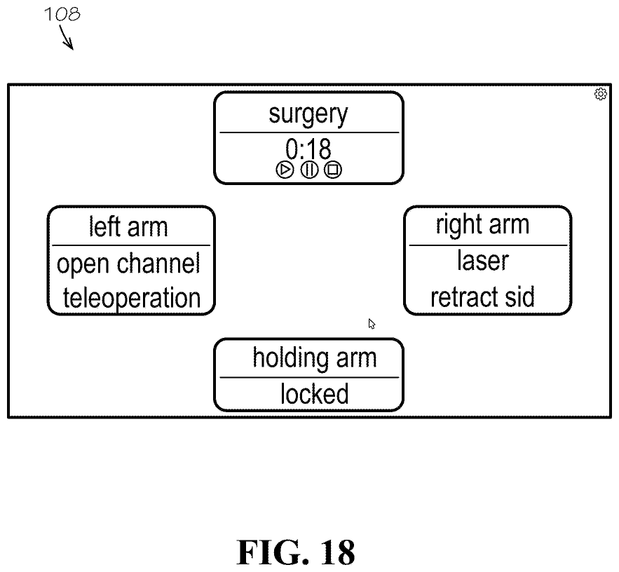

Referring to FIGS. 16 and 17, in one embodiment, the physician input console 40 provides an interface for the surgeon. This interface may include a screen 102, one or more buttons 104, speakers, or light indicators 106. The screen 102, which may include a touchscreen and may be operable through a drape, may display the system state, the duration of the surgical procedure, the state of the holding arm 12, or indicate which instrument is in the left side of the system or the right side of the instrument. One embodiment of a graphical user interface 108 that may be displayed on the screen 102 is shown in FIG. 18. This interface 108 may also display recoverable or non-recoverable fault information and instructions to the user for resuming. The interface 108 may provide graphical instructions for re-registering the input controls 42. The physician input console 40 may emit an audible signal prior to the initiation of new manipulator motion, which may serve as a safety feature to detect an intentionality subsystem failure and alert the operating room staff. The one or more buttons 104 or touchscreen 102 may provide for inputs that can begin the surgery, pause the surgery, or end the surgery. The screen 102 may provide instructions for proper system setup, breakdown, and operation. The screen 102 or speakers may alert the user if a system step is performed out of order, such as if the actuation unit 20 is unplugged prior to removing the instrument cartridges 410.

Referring to FIG. 17, in one embodiment, the physician input console 40 provides an input control adjuster 110 that, when rotated, provides side-to-side adjustment of the first or second input controls 42, 44 within one or more horizontal tracks 112. This ergonomic adjustment provides for surgeon comfort across anatomic variation and preferences. The input control adjuster 110 may include a knob, dial, or other type of input control adjuster.

### Surgeon Input Device Re-Registration Process

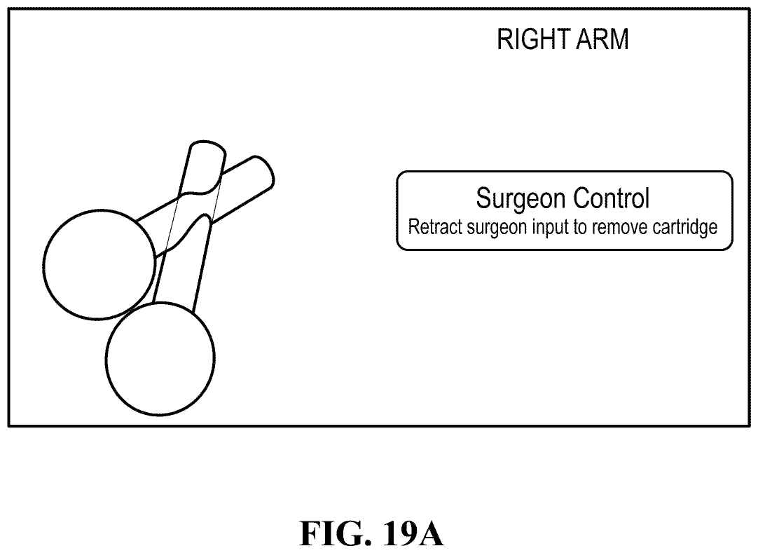

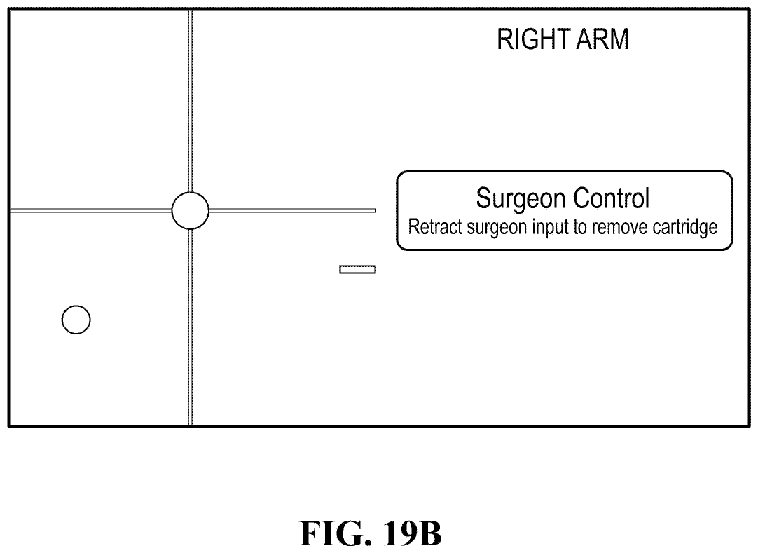

The first or second input controls 42, 44 may become un-registered with the concentric tube manipulators if they move when intentionality is not detected, when the surgery is paused, when a fault is detected, or before or after the surgery has begun. Re-registering instructions are provided on the screen 102. Re-registering instructions may include a real-time transparent three-dimensional overlay of the current position or orientation of the first or second input controls 42, 44 on top of the desired/re-registered pose of the input controls 42, 44 and a progress indication displaying re-registration progress. Similarly, the re-registration instructions may include a two-dimensional target marker and a current two-dimensional position marker along with a progress indication. Potential embodiments of re-registration instructions on the graphical interface are shown in FIGS. 19A and 19B.

### Surgeon Workstation Ergonomics

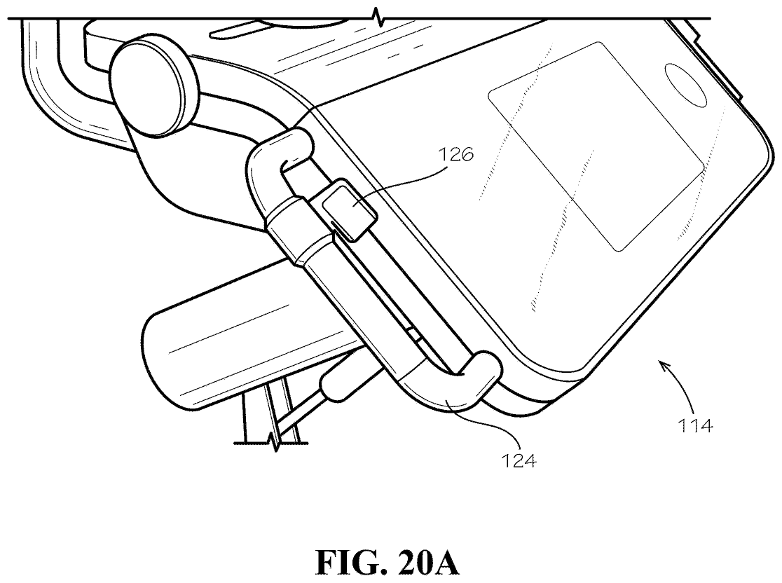

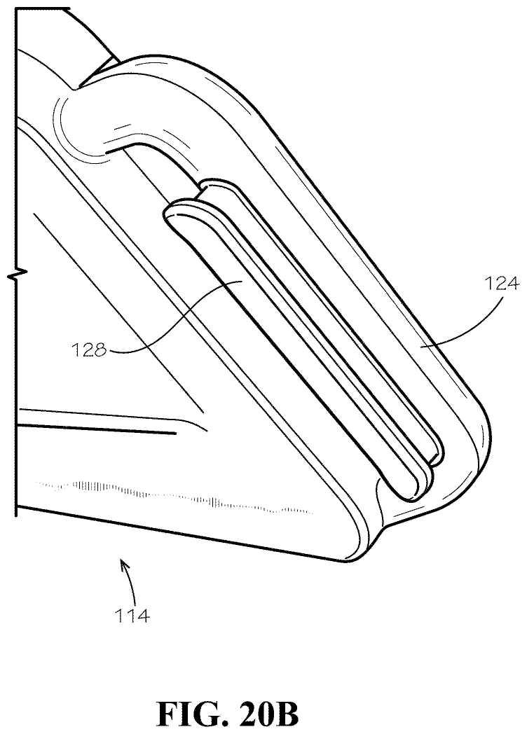

In some embodiments, the robotic system 10 may impose one or more anatomic constraints on a surgeon using the system 10. These anatomic constraints may create short-term or chronic surgeon discomfort, as some surgical procedures may be long, and a surgeon may perform some procedures repetitively. The system 10 provides a physician input console 40 that can adjust the position of the top tray 114 or the first or second input controls 42, 44 such that the surgeon operator can stand or sit when using the input controls 42, 44. In one embodiment, the four-bar linkage 116 enables this movement, and the gas spring 118 provides gravity compensation so that the tray does not fall under gravity. The design of the four-bar linkage 116 moves the top tray 114 towards the surgeon as it moves downwards, which creates additional foot space on the ground when the surgeon is in a seated position. In some embodiments, the physician input console 40 may not impose specific foot position requirements on the surgeon to operate any of the surgeon controls of the physician input console 40. In one or more embodiments, the base 120 of the physician input console 40 is configured as an “X” or “U” shape to increase available foot space for the surgeon while still providing a large wheel base for stability of the physician input console 40 during transport. The base 120 may include one or more casters 122 or other types of wheels for transporting the physician input console 40. The surgeon or another operator may adjust the position of the top tray 114 by depressing an input either in the side handle 124 or under the top tray 114. In some embodiments, this input may include a toggle-style input 126, as shown in FIG. 20A. In other embodiments, the input may include a push-style input 128, as shown in FIG. 20B.

Prior art surgical robotic systems often require specific elbow, head, forehead, or forearm positions at the physician interface. Often, the surgeon controls will only become active when a sensor measures specific positioning of the elbow, head, forehead, or forearm. In certain embodiments, the physician input console 40 does not impose elbow or forearm positional constraints on the surgeon. Prior art surgical robotic systems may provide physician interfaces that restrict the surgeon's view of the operating theater. The surgeon's view may be restricted by a large screen in front of them or by requiring them to look into eyepieces integrated into the physician interface. In one embodiment, the physician input console 40 provides an unobstructed view of the operating theater while operating the first or second input controls 42, 44. Prior art surgical robotic system physician interfaces typically prevent late-term pregnant surgeons from operating the surgeon controls due to the anatomic constraints imposed by the physician interface. The physician input console 40 may impose no anatomic constraints that would prevent the use by a late-term pregnant operator.

### Surgeon Input Device Ergonomics

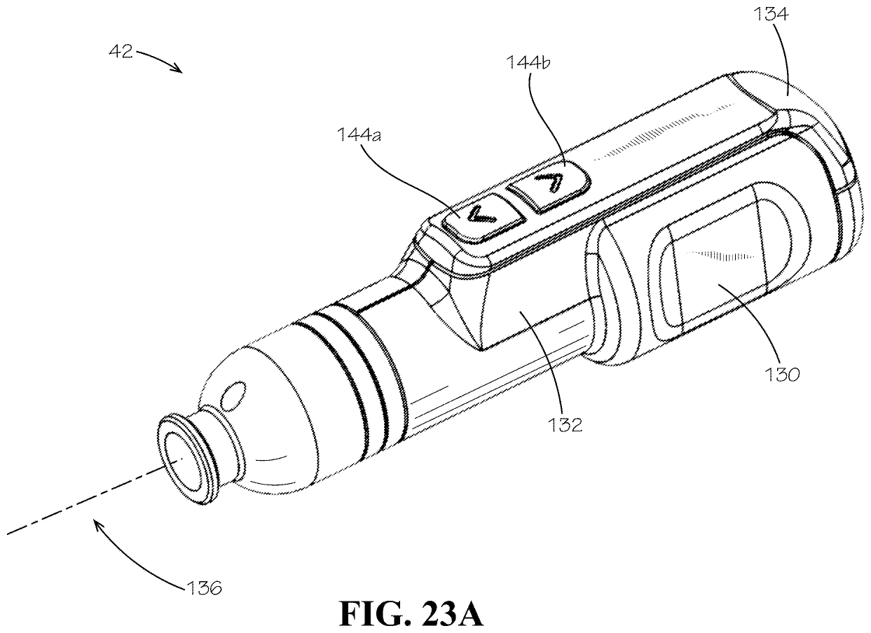

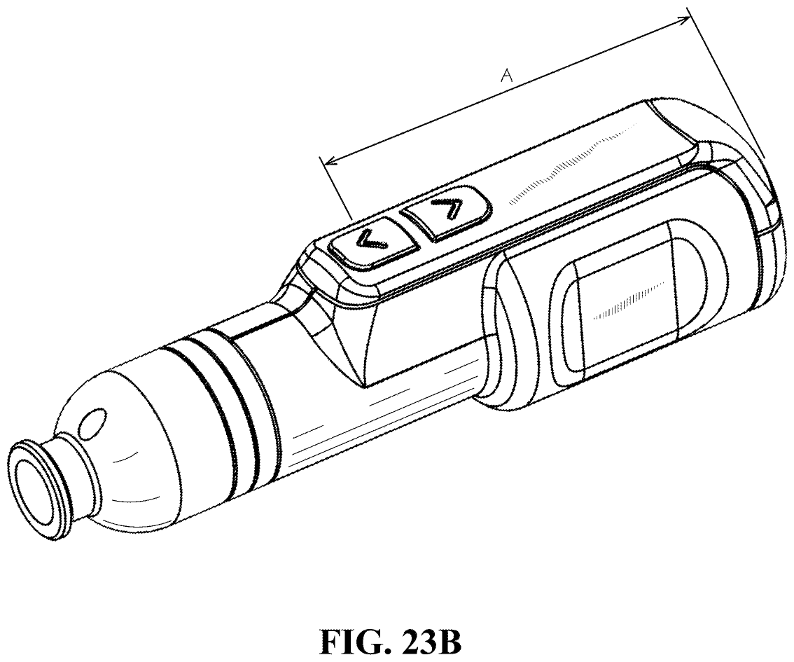

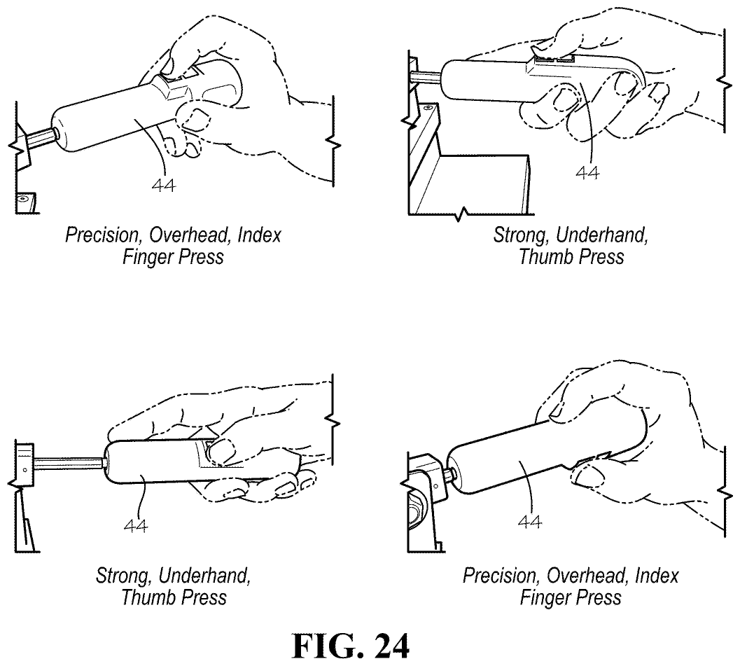

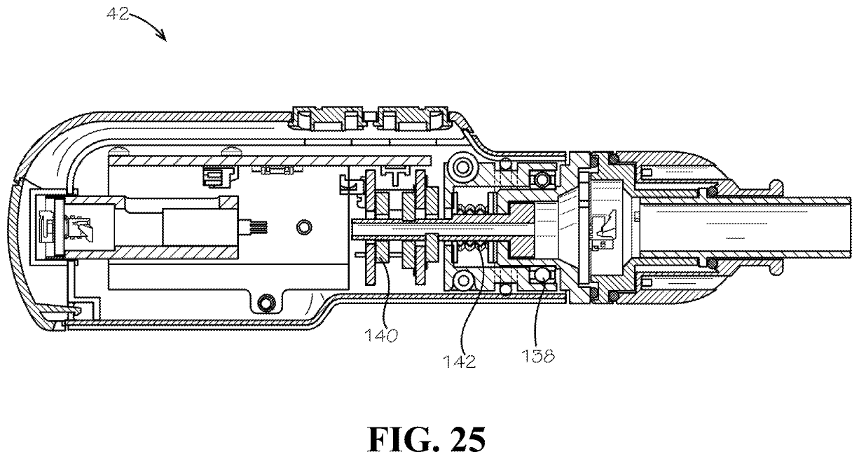

While the following disclosure discusses subject matter in reference to the first input control 42, such discussion is applicable to the second input control 44. Referring to FIGS. 23A and 23B, in one embodiment, the first input control 42 handle is cylindrically shaped to enable a precision grip a full grasp, an overhand grip, or an underhand grip. The first input control 42 may be shaped or configured to enable a tool button press by the index finger or by the thumb, each of such grip are shown in FIG. 24. The cylindrical shape may enable precise or strong/full grasps. The first input control 42 may be shaped such that the gripping surfaces are contained within bounding cylinders of 1 inch (approx. 2.54 centimeters) in diameter and 1.5 inches (approx. 3.81 cm) in diameter. The first input control 42 handle may include one or more flats 130 or other orienting features like a raised edge 132 so that the surgeon can tactilely orient the handle in their hand without looking down at the handle. This makes surgery safer by enabling the surgeon to keep their eyes fixed to the operating room display 60. The rounded top 134 of the handle, in some grips, may be seated within the palm of the surgeon (see FIG. 24). Securing this rounded top 134 within the palm may create a strong and stable support structure within the surgeon's hand for control of the first input control 42. The handle may include a roll degree-of-freedom around its main axis 136. This may be enabled by an internal bearing 138 depicted in FIG. 25. The roll degree of freedom may be sensed by one or more angular position sensors 140. In one embodiment, these sensors 140 may include potentiometers, and redundant angular position sensors 140 may be provided in the first input control 42 for additional safety. The first input control 42 may be configured to provide damping friction on each degree-of-freedom. In the handle of the first input control 42, this may include the friction-addition feature 142. The first input control 42 can be let go and re-gripped. The first input control 42 may be lightweight or statically balanced so that it may not move once released.

### Surgeon Input Device Tool Buttons

As seen in FIG. 23A, in some embodiments, the first input control 42 may include two integrated tool buttons 144a, 144b. The system 10 can deliver an array of tools 46 including gripping tools, tools that surround tissue and grasp it such as a basket or a snare, or energy-delivery tools such as lasers or electrosurgical probes, among others. The first input control 42 may include two tool buttons 144a and 144b for actuation of these tools 46. For tools 46 that can extend/retract, the distal (i.e. furthest away from the surgeon) button 144a may include an arrow pointing away from the surgeon. The button 144a may extend the tool 46 out of the manipulator when pressed and held. The proximal button 144b may include an arrow pointed towards the surgeon. The button 144b may retract the tool 46 into the manipulator tip when pressed and held. One of the two tool buttons 144a, 144b may be colored yellow and activate the electrosurgical cut input. The other of the two tool buttons 144a, 144b may be colored blue and activate the electrosurgical coagulation input. Depression of these buttons may open or close electrical contacts or create another electrical information signal that can serve as an activation input to an electrosurgical generator. This electrosurgical generator may be included with the system 10 or may interface with the system 10. These tool buttons 144a, 144b may be between 40 and 80 millimeters from the end of the first input control 42 handle. This measurement is denoted by length A in FIG. 23B.

### Surgeon Input Device Intentionality Sensing

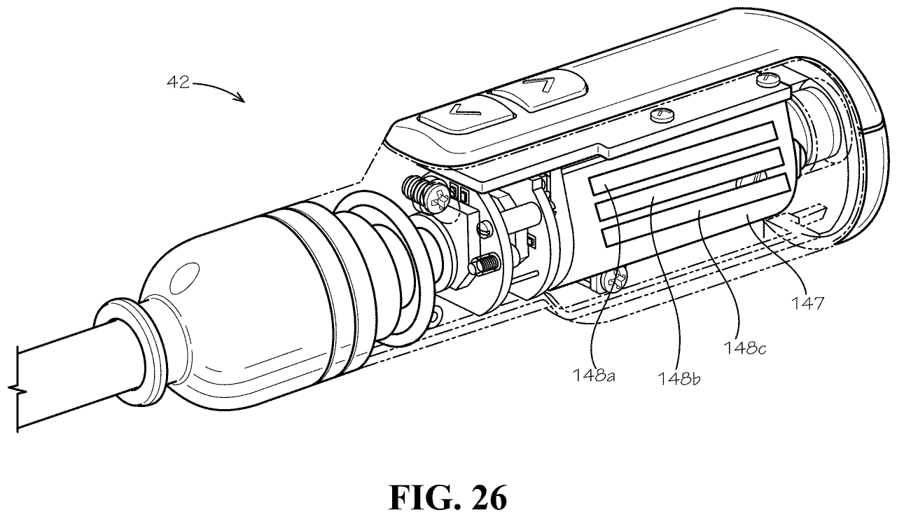

Referring to FIG. 26, in one embodiment, the first input control 42 handle includes an internal flexible circuit board 184 wrapped around the longitudinal axis and is capable of sensing capacitive changes on several channels (146a, 146b, 146c) due to touch, including through a drape and with gloves on. The purpose of this capacitive sensing is to determine intentionality. In one embodiment, the capacitive touch sensors provide seven independent channels around the longitudinal axis. In the event that the first input control 42 is accidentally bumped or hit by a cable, or the physician input console 40 is accidentally bumped, these movements will not be conveyed to the concentric tube manipulators. Intentionality sensing is an important safety consideration, but this embodiment may place no additional unergonomic constraints on the surgeon often seen in other systems (for example, elbow position, forehead position). The control system may include multiple channels 146a, 146b, 146c, potentially non-adjacent channels to be active in order to convey surgeon control motion to the manipulators at the surgical site. This may provide further safety so that if the first input control 42 is bumped on one of its sides, and not grasped on both sides, motion will not be conveyed to the concentric tube manipulators.

### Physician Interface in Sterile Field

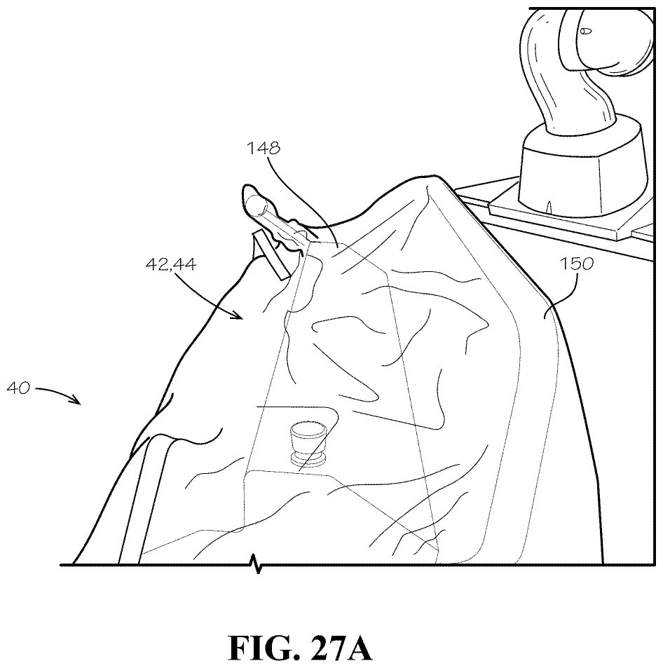

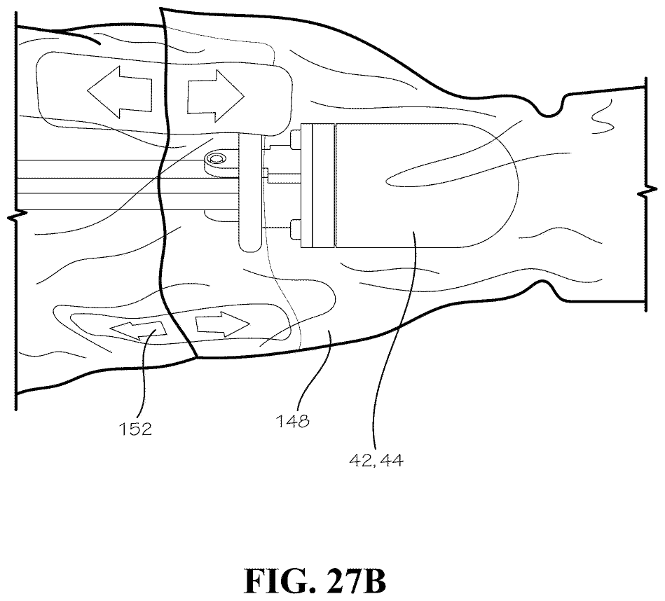

Prior art surgical robotic systems typically require that the physician interface be used outside of the sterile field. The physician input console 40 may be configured to be used in the sterile field, if desired. Referring to FIGS. 16 and 27A, in some embodiments, the drape bar 150 allows for a drape 148 to be “tented” over the first or second input controls 42, 44 so that they do not interfere with the drape 148 during motion. After the drape 148 is tented over the drape support bar 150, the drape 148 may fall towards the floor, covering about halfway between the top tray 114 and the floor. As seen in FIG. 27B, in one embodiment, the drape 148 may extend over both of the first or second input controls 42, 44 and may include a tear tab 152 and a coated wire at the interface between the handle and the main input control 42, 44 shaft on both sides. In one embodiment, this coated wire can be secured and the tear tab 152 can be torn such that the handle portion of the drape 148 becomes independent of the rest of the drape, can rotate without the need for a joint limit, and without bunching up the drape 148, and while still maintaining a sterile interface.

### Articulated Arm Base

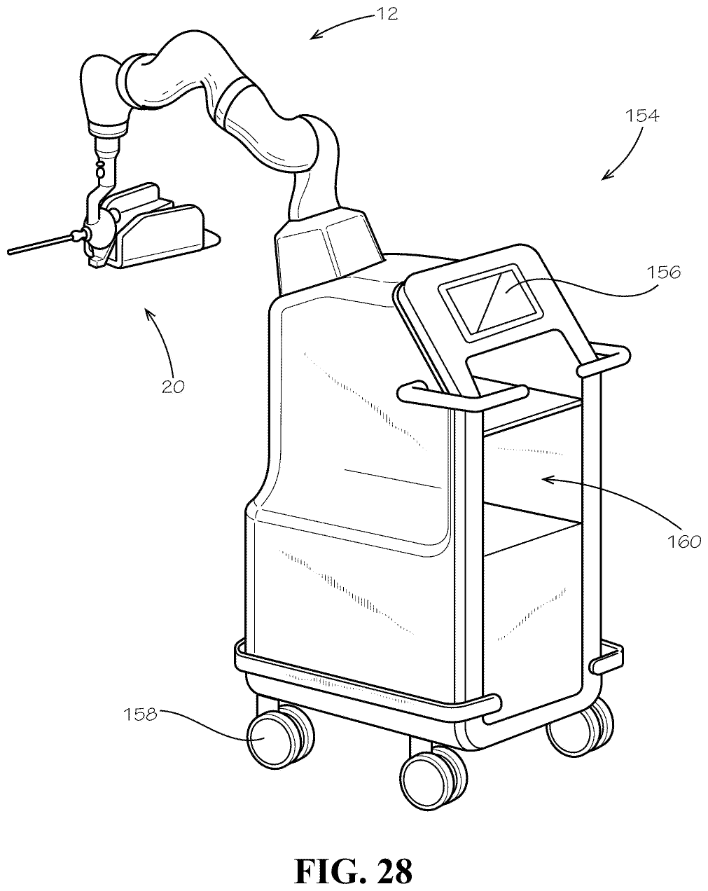

Referring to FIG. 28, in some embodiments, an articulated arm base 154 may provide a screen or touchscreen 156 with instruction for setup and breakdown. This screen 156 may enable the user to command the articulated holding arm 12 to be extended for drape application. The screen 156 may further allow for the user to command calibration of the holding arm 12 and enable the application of the caster brake 158. The screen 156 may include the display 60 or the screen 102. In some embodiments, the articulated arm base 154 may provide a vertical adjustment, either actuated or passive, that enables the articulated holding arm 12 to be positioned further or closer to the ground. The articulated arm base 154 may include storage 160 for other system equipment such as the holding arm electronic controller, power supplies, or an isolation transformer. The articulated arm base 154 may include space for endoscopy equipment and an electrosurgical generator.

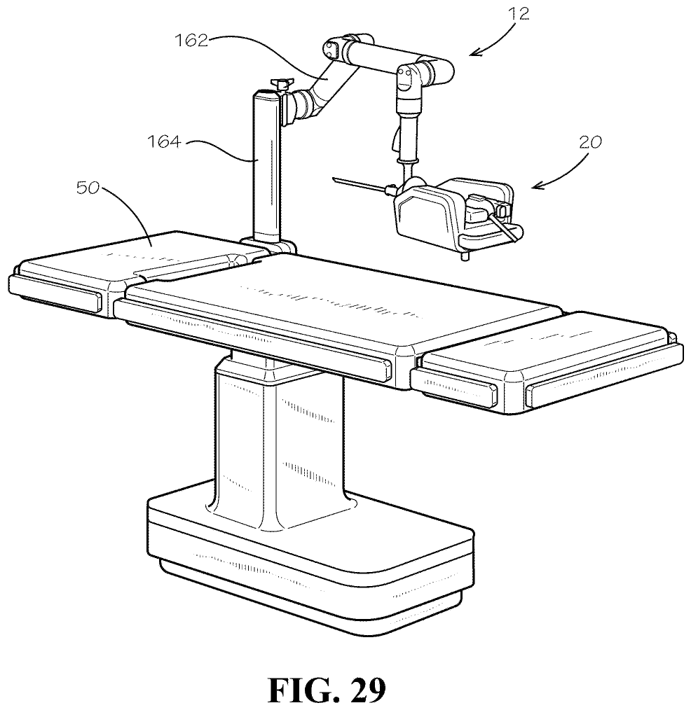

Referring to FIG. 29, the articulated arm base 162 may be mountable to the rails of an operating table 50. This holding arm 12 may be actuated or passive with braking features. This mounting may include a vertical bar 164 that may allow for adjustment of the vertical positioning of the articulated holding arm 12 to adapt to patient positioning and patient anatomy.

In one embodiment, the articulated arm base 162 may include a cart similar to the cart of FIG. 28, but may include mounting features similar to operating room rails, so that an articulated holding arm 12 can be disconnected and re-mounted in many possible configurations. This enables additional versatility for patient positioning and clinical indications for the system 10.

### Emergency Stopping Devices

Referring to FIG. 17, in one embodiment, the physician input console 40 include an emergency stopping device 166 which may immediately prevent holding arm 12 motion (if actuated), manipulator motion, or electrosurgical output. Referring to FIG. 29, the articulated arm base 154 may include an emergency stopping device near the touchscreen 156 which performs the equivalent function as the emergency stopping device 166 on the physician input console 40. Emergency stopping devices 166 are a safety feature for the operating room staff or surgeon if the system 10 is behaving in an unexpected or unsafe way.

### Motor Control Safety Supervisory System

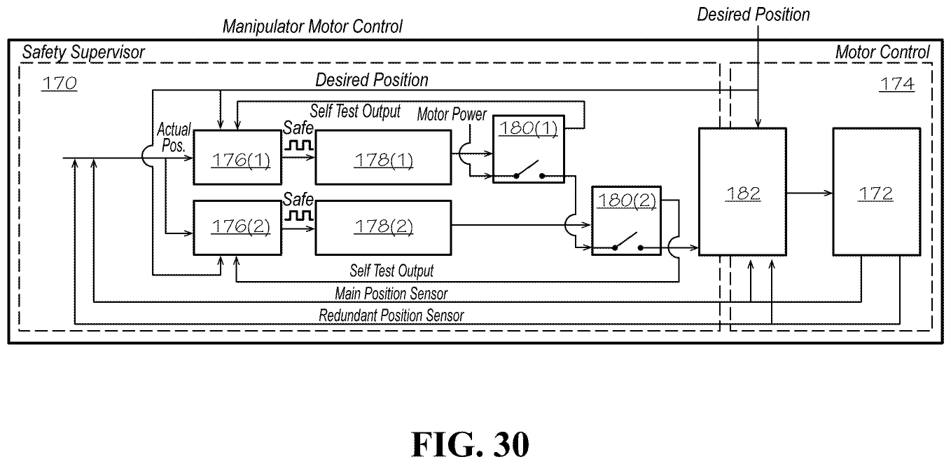

Referring to FIG. 30, in one embodiment, the actuation unit 20 includes a safety supervisory system 170. The purpose of this system 170 is to stop one or more motors 172 in a motor system 174 in the event that a safety limit is breached. This safety supervisory system 170 features redundant computing elements (such as safe central processing units 176(1) and 176(2)) that read in motor position and velocity values and compares it to their commanded position and velocity values. If these values are within their safety limits, the computing elements sends a heartbeat signal to a heartbeat monitor integrated circuit chip (such as heartbeat monitor 178(1) or 178(2)) that latches a safety relay (such as safety relay 180(1) or 180(2)) that is in communication with one or more motor drivers 182 that control motor power to the actuators. In the event that a computing element becomes stuck or non-responsive, the heartbeat monitor 178(1) or 178(2) will quickly latch the relay 180(1) or 180(2) so that the motors 172 stop. Without power, the safety relays 180(1) or 180(2) are open so that no power can reach the motors 172. This basic pathway is repeated in parallel with redundant sensors so that the safety supervisory system 170 has internal redundancy. During startup and/or intermittently during operating, the relays 180(1) or 180(2) are self-tested to ensure that they can still open and close as intended. This safety supervisory system 170 may experience three independent failures to occur simultaneously and without detection to violate safety, which is extraordinarily unlikely to occur. This safety supervisory system 170 may be embedded within the actuation unit 20 itself.

### Cartridge Sensing Subsystem

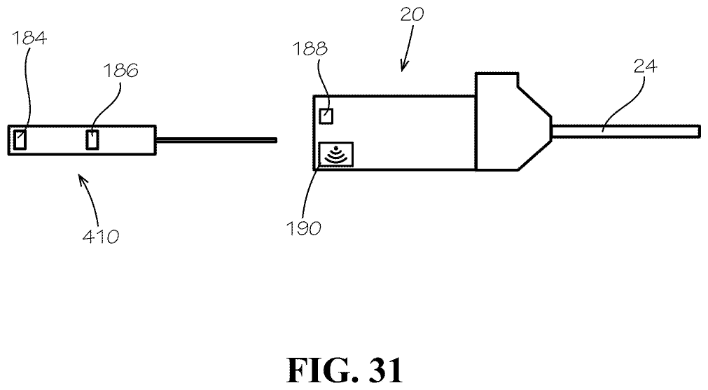

Referring to FIG. 31, in one embodiment, an instrument cartridge 410 may include a cartridge magnet 184 and an RFID tag 186. The RFID tag 186 may include information on the tool type of the instrument cartridge 410, information on if the instrument cartridge 410 has previously been used, and a password that is encrypted by the system 10 which may prevent the RFID tag 186 from being written by a third party or may prevent usage of fraudulent instrument cartridges 410. Once the instrument cartridge 410 is inserted, the system 10 may validate that the instrument cartridge 410 has not been previously used and may adjust the manipulator kinematics for the inserted tool 46. The cartridge sensing subsystem may also utilize embedded magnets and hall sensors 188 to detect when the instrument cartridge 410 is fully inserted. Motion of the actuators may be prevented until the instrument cartridge 410 is verified to be valid and it is confirmed to be fully inserted. The RFID module 190 can modulate its read power to differentiate between which cartridge 410 is inserted on the left side of the actuation unit 20 and which cartridge 410 is inserted on the right side of the actuation unit 20. This subsystem enables the building out of additional instruments which only require a software update on the rest of the system 10 to use. In one embodiment, the cartridge chipset 441 may include the cartridge magnet 184 or the RFID tag 186.

### Motion Through Drape: Motor Pack

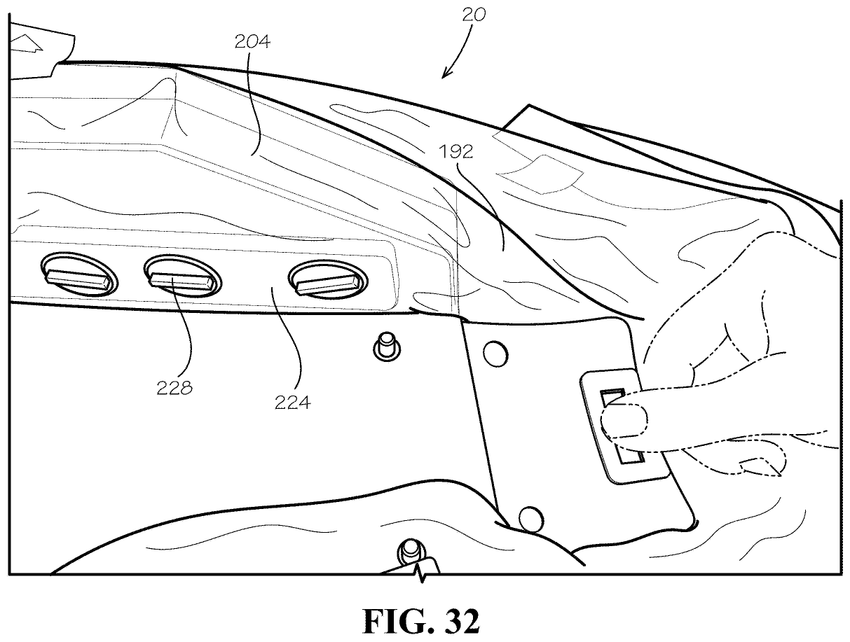

Referring to FIG. 12, in one or more embodiments, the actuation unit 20 may include a drape plate 224 with integrated couplings 228. This plate 224 may provide motor motion through a sterile interface so that the manipulators embedded in the sterile instrument cartridges 410 can be actuated. The drape plate 224 snaps to the side wall 204, 206 of the actuation unit 20 so the nursing staff can easily assemble during setup. The motor output couplings, which may be located immediately behind the drape plate couplings 228, may include an axial spring that allows the drape plate 224 to be snapped to the wall without its couplings aligned with the motor couplings. During startup, the actuation unit 20 may spin each of its axes 360 degrees, which enables the motor output couplings to spring into the correct mating position with the drape plate 224. The couplings are then held flat to enable instrument cartridge 410 insertion utilizing the flathead interface. FIG. 32 depicts the drape plate 224 integrated into the actuation unit drape 192.

### Articulated Holding Arm Unlock

In one embodiment, the articulated holding arm 12 includes a gripping handle for a strong power grip. The diameter of the handle may be between 1 and 4 inches (approx. 2.54 cm to 10.16 cm). The handle may include an unlock mechanism. In the case of an actuated holding arm 12, the unlock mechanism may include a button or switch contact which may be connected to the holding arm control system. The unlock mechanism may include multiple buttons that enable different types of motions, for example motion only along the endoscope axis, heavily damped motion, lightly damped motion, only translation (no rotation), only rotation, or only rotation about a selectable center of rotation. In the case of a passive holding arm 12, the unlock mechanism may include a mechanism that unlocks all of the joints of the articulated holding arm 12. The handle may be located near the center of mass of the actuation unit 20 so that it can more easily be manipulated without the surgeon operating room staff feeling large torques on their hand.

### Instrument Cartridge Deliverable Tool Interface

Referring to FIG. 14, in one embodiment, the instrument cartridges 410 may include user access to the inner lumen of the concentric tube manipulator from the back of the instrument cartridge 410. A moving rod 490 may provide the user access. This moving rod 490 may move with the innermost tube of the concentric tube manipulator. The distal tip of the innermost tube of the concentric tube manipulator may include the tip of the instrument the surgeon sees in the surgical field. If a deliverable tool 46 or instrument is delivered through this rod 490 to the tip of the manipulator and secured to the movable rod 490, this instrument may move with the manipulator tip. The movable rod 490 may feature a collet mechanism or similar mechanism for axially holding a deliverable instrument such as a laser fiber or an electrosurgical probe. This may enable the usage of existing probes or devices that need not be provided pre-assembled within the instrument cartridge 410. It also enables the possibility of re-using these instruments if they are capable of being re-processed and saving hospital expense for the procedure.

### Non-Annular Concentric Tube Manipulator Tip

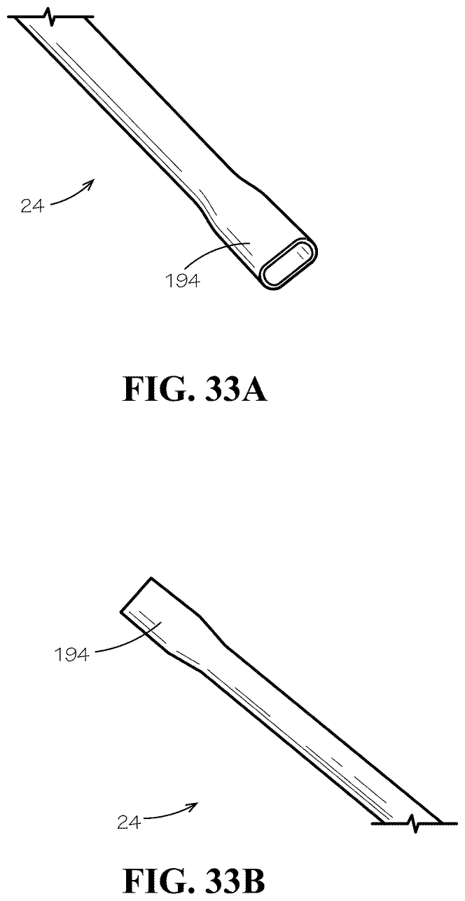

Referring to FIGS. 33A and 33B, in one embodiment, the tip 194 of the innermost tube of the concentric tube assembly 24 is shown. For some tool configurations, it is useful to shape the tip 194 in a non-annular cross-section. The concentric tube manipulator may include nitinol, a material that can be temperature set into different shapes. This non-annular shape may be useful for tools 46 that have a non-annular cross-section. These tools 46 may still be able to translate through the tube, but will be prevented from rotating with respect to the manipulator tip 194. This means that the tool 46 may provide relatively high torsional stiffness, which may be useful in many surgical contexts. This could, for example, make an electrosurgery probe stiffer and more rugged, or make a grasping or retracting instrument more rugged and able to place higher forces on tissues without deflecting, enabling a more useful retraction instrument.

Thus, although there have been described particular embodiments of the present invention of a new and useful SYSTEM FOR PERFORMING MINIMALLY INVASIVE SURGERY, it is not intended that such references be construed as limitations upon the scope of this invention except as set forth in the following claims, or in additional claims provided in future applications claiming priority to this provisional.

What is claimed is:

1. An apparatus for performing surgery, comprising:
a holding arm;
a holding arm interface detachably secured to the holding arm;
an actuation unit detachably secured to the holding arm interface;
an endoscope sheath assembly comprising an inner sheath and an outer sheath detachably secured to the holding arm interface opposite the actuation unit.

2. The apparatus of claim 1, wherein the holding arm comprises an articulated holding arm.

3. The apparatus of claim 1, further comprising:
a removable cartridge disposed on the actuation unit, the removable cartridge comprising a concentric tube array extending through the holding arm interface and into the endoscope sheath assembly.

4. The apparatus of claim 3, further comprising:
a channel disposed inside the inner sheath, wherein the concentric tube array is positioned inside the channel.

5. The apparatus of claim 4, further comprising an interface mount on the holding arm interface, wherein the interface mount provides a connection between the holding arm and the holding arm interface.

6. The apparatus of claim 4, wherein the actuation unit is detachable relative to the holding arm interface along a longitudinal axis.

7. The apparatus of claim 6, wherein the endoscope sheath assembly is detachable relative to the holding arm interface along the longitudinal axis.

8. The apparatus of claim 7, wherein the concentric tube array is at least one of:
axially moveable along the longitudinal axis; or
angularly moveable about the longitudinal axis.

9. The apparatus of claim 8, wherein the holding arm interface comprises a base plate and a shell, wherein the base plate is angularly moveable relative to shell about the longitudinal axis at a rotating joint.

10. The apparatus of claim 9, wherein the actuation unit is angularly moveable relative to the holding arm interface via the rotating joint.

11. The apparatus of claim 10, further comprising a brake disposed on the holding arm interface, wherein the brake is configured to selectively angularly lock the actuation unit at a desired angular orientation relative to the holding arm interface.

12. The apparatus of claim 11, further comprising a handle on the holding arm interface.

13. The apparatus of claim 12, further comprising a first button on the handle, wherein the first button is configured to selectively operate a feature of the system.

14. The apparatus of claim 3, the cartridge further comprising a plurality of cartridge couplings.

15. The apparatus of claim 14, the actuation unit further comprising a plurality of actuation couplings, wherein each actuation coupling corresponds to a cartridge coupling.

16. The apparatus of claim 15, further comprising:
a linear cartridge slot defined on the cartridge; and
a coupling flange protruding from each actuation coupling,
wherein each coupling flange is received in the linear cartridge slot when the cartridge is inserted into the actuation unit.

17. The apparatus of claim 16, further comprising a linear coupling slot defined in each cartridge coupling, wherein each cartridge coupling receives a corresponding coupling flange when the cartridge is fully inserted into the actuation unit.

18. The apparatus of claim 17, further comprising:
a plurality of drive motors disposed on the actuation unit, wherein each drive motor is linked to an actuation coupling, and wherein each drive motor is operation to control rotation of a corresponding cartridge coupling.

19. The apparatus of claim 18, further comprising:
a chipset disposed on the cartridge, wherein the chipset comprises memory configured to store information about the cartridge.

20. The apparatus of claim 19, further comprising a latch on the actuation unit, wherein the latch is selectively operable to secure or to remove the cartridge from the actuation unit.
